(19) United States
(12) Patent Application Publication
(10) Pub. No.: US 2018/0333318 A1
(43) Pub. Date: Nov. 22, 2018

(54) Positioning System for Robotic Knee Testing Apparatus and Method of Using Same

(71) Applicant: ERMI, Inc., Atlanta, GA (US)

(72) Inventors: Thomas P. Branch, Atlanta, GA (US); Shaun K. Stinton, Chamblee, GA (US); Edward Dittmar, Marietta, GA (US); Nathaniel K. deJarnette, Lilburn, GA (US); T. Christopher Madden, Atlanta, GA (US)

(21) Appl. No.: 15/601,410

(22) Filed: May 22, 2017

(51) Int. Cl. A61G 13/12; A61B 34/30; A61G 13/00; B25J 11/00; B25J 9/00

(52) U.S. Cl. CPC A61G 13/1245; A61B 34/30; B25J 9/0027; B25J 11/009; A61G 13/0018

(57) ABSTRACT

A robotic knee testing apparatus including a robot configured to support a leg of a patient, a patient support configured to support the patient thereon, and a positioning system adjustably connected to the patient support and movable relative to the patient support, wherein the positioning system is movable to adjust a position of the robot so as to allow the patient to be situated in an orthostasis position between a distal edge of the patient support and the robot. The robotic knee testing apparatus can also include a positioning system that is adjustably connected to the patient support and movable relative to the patient support to adjust a vertical position of the robot relative to the patient support.

### BACKGROUND

### 1. Field of the Disclosure

The disclosure generally relates to robotic knee testing and evaluation, and more particularly to a positioning system for a robotic knee testing apparatus and to a method of using the positioning system.

### 2. Description of Related Art

The knee joint is composed of the femur or thigh bone, the tibia or shin bone, and the patella or knee cap. The bones are connected by fibrous structures called ligaments, which allow a certain amount of “joint play” or motion to exist between the bone structures. When this joint play is increased or decreased, an abnormal or pathological condition exists in the knee. Attempts have been made in the past to quantify this increase or decrease in joint play of the knee with limited success.

Knee injuries often cause damage to one or more of the structures that form the knee joint. Such injuries typically cause an increase in joint play or motion of the knee. A patient may interpret an increase in joint play as a sensation that the knee is slipping or coming out of joint. In other words, this sensation may be described by the patient as the feeling of joint instability. Knee instability may be related in part to an increase in the length of the ligaments that connect the bones together, an increase or change in compliance (elastic resilience or stretchiness) of the ligaments, or both. Knee instability may also be related in part to the shape and size of the joint bones. The degree or likelihood of the knee joint bones actually coming out of joint or becoming unstable is related to the amount of stretch or increased length of each knee ligament, the number of knee ligaments involved, and the existence of damage to one or more other support structures of the knee joint, such as the joint bones themselves, the menisci, or the like. Accurate measurement of an increase in ligament length can be critical to restoring a patient's injured or damaged knee to as close as possible to its original functional and anatomical structure and condition.

For the most part, knee injuries and ligament damage have been diagnosed using only manual tests. These tests are performed by doctors or other medical personnel, i.e., clinicians, on the patient in order to detect and measure joint play to diagnose damage to the knee ligaments or other knee joint support structures. There are a number of commonly known manual tests used to evaluate increased joint play, which is usually associated with an anterior cruciate ligament (ACL) tear. These tests include the Lachman's test, the Pivot Shift test, and the Anterior Drawer Test. Because these tests are performed manually by individual medical personnel, these tests naturally are limited by the specific clinician's subjective evaluation. The subjective nature of the tests may hinder the precision or accuracy of any diagnosis of the extent of ligament lengthening, the change in ligament compliance or elastic resilience, i.e., stretchiness, or both.

In order for a clinician to diagnose an injured ACL, the clinician must determine whether the knee feels “abnormal.” The accuracy of an ACL injury diagnosis provided by a clinician using currently known manual tests depends on the skill and experience of the clinician and their subjective determinations. A misdiagnosis can lead to unnecessary treatment or unnecessary delay in treatment, which may result in an increased risk for further injury or damage to the patient's knee joint.

There are also manual tests for the lateral collateral ligament (LCL), medial collateral ligament (MCL), and posterior cruciate ligament (PCL). Each manual test relies on grading the degree of length increase in the ligament based on relative increase in joint play into three Grades or categories. There is no effort to grade the compliance or elastic resilience, i.e., stretchiness, of the ligaments using these manual tests. However, an expert clinician may describe the ligament in terms of its subjective feel to the clinician. Also, a knee joint may have injury or damage to more than one ligament or structure. The more ligaments and structures of the knee joint that are damaged, the more complex it is for the clinician to perform a manual knee examination. This can make the diagnosis less accurate and less precise.

Clinicians and surgeons manually examine the injured knee joint for altered or increased joint play. However, due to the variability in size of the patient, size and experience of the surgeon, and the potential degree or subtlety of an injury, consistent and reproducible reports of joint play between surgeons is not possible. Many reports have documented that, whether diagnosis is performed manually or even with manual arthrometers, the manual application of torque to the knee joint varies widely between clinicians. This results in inconsistencies in the examination of joint play.

Others have attempted to reduce the manual nature of such joint tests and to instrument the knee joint during testing. The objective has been to mechanically or objectively quantify or measure a change in the structure of the knee after ligament damage. Several devices have been developed in attempting to more accurately quantify the extent of injury or relative displacement and compliance of a ligament in the knee. In one example, such devices have been developed by Medmetric Corp. These devices include the KT-1000 and KT-2000 models (hereinafter “KT”). The KT devices are intended to measure the anterior-posterior translation of the tibia with respect to the femur along the y-axis. The KT devices attach to the patient's tibia during testing.

The KT devices attempt to quantify the findings achieved by a clinician performing the Lachman's test and the Anterior Drawer Test. Force is applied to a handle on the device, which measures the force and delivers the amount of applied force to the clinician using sounds, such as a low pitched sound for a 15-pound force and a higher pitched sound for a 20-pound force. The applied force in the KT devices pulls anteriorly along the y-axis through a strap that wraps underneath the patient's calf. The translation is determined using a technique that measures the relative motion between a pad placed against the anterior tibia and a pad placed against the patella. The KT devices do not measure relative displacement or compliance in any of the other degrees of freedom in the knee. Also, quantified results from using the KT devices have not been correlated with patient satisfaction. In contrast, the subjective Pivot Shift test has been correlated with patient satisfaction.

Other devices are also known and include the Stryker KLT, the Rolimeter, and the KSS system. These known devices use similar mechanisms to attempt to quantify the normal amount of joint play or motion between the femur and tibia in the knee joint, as well as any increased joint play or motion in the joint associated with ligament lengthening and damage. The applicant of the instant application has developed robotic knee testing (RKT) apparatuses, the basics of which are disclosed and described in U.S. publication nos. 2012/0046540 and 2014/0081181. Each apparatus, in part, utilizes motors to perform knee movements during testing and employs sensors to measure degree of relative movement of the structures in the knee joint. Portions of the knee and leg can be stabilized or moved, as needed during testing.

Patients, especially individuals with limited mobility, encounter difficulty when positioning themselves onto a patient support of a robotic knee testing apparatus such as those described in U.S. publication nos. 2012/0046540 and 2014/0081181. The patient is required to sit on the patient support and pivot their body while lifting their legs up and over the robot. Once in this position, the patient lowers their legs into the robot support. It can be difficult from some patients to climb up onto the patient support. It can also be difficult form many patients to maneuver their legs up, over, and onto the robot.

These existing robotic knee testing apparatuses are typically limited to testing the knees when in a specific flexion angle at 30 degrees of knee flexion. Testing at a fixed or single knee flexion angle can result in incomplete testing of the ligaments or may not be suitable for all patients. Ligaments may exhibit greater resistance to rotation at certain degrees of flexion and less at others.

These issues with existing instrumented devices and robot apparatus may lead to a limited or incomplete perspective of the patient's joint damage.

### SUMMARY

In one example according to the teachings of the present invention, a robotic knee testing apparatus can include a robot configured to support a leg of a patient, a patient support configured to support the patient thereon, and a positioning system adjustably connected to the patient support and movable relative to the patient support. The positioning system is movable to adjust a position of the robot so as to allow the patient to be situated in an orthostasis position between a distal edge of the patient support and the robot.

In one example, the positioning system of the robotic knee testing apparatus can be movable to adjust a vertical position of the robot.

In one example, the positioning system of the robotic knee testing apparatus can further include a column lift supporting the robot, the column lift being operable to adjust a vertical position of the robot relative to the patient support.

In one example, the column lift of the robotic knee testing apparatus can include an inner segment and an outer segment telescopically connected relative to one another.

In one example, the positioning system of the robotic knee testing apparatus can be movable to adjust a horizontal position of the robot toward and away from the patient support.

In one example, the positioning system of the robotic knee testing apparatus can further include a guide such that a portion of the positioning system is slidable relative to the patient support.

In one example, a robotic knee testing can include a guide that includes at least one guide rod with a first end fixed to a base of the positioning system and a second end.

In one example, a robotic knee testing apparatus can include at least one guide rod, with a second end of at least one guide rod being slidably coupled to a portion of the patient support.

In one example, a robotic knee testing apparatus can include a guide with a pair of the guide rods.

In one example, the positioning system of the robotic knee testing apparatus can further include a locking mechanism to lock the positioning system in a selected position so as to fix the position of the robot relative to the patient support.

In one example, a robotic knee testing apparatus can include a step tread attached to the patient support and located between a patient support and the positioning system.

In one example, a robotic knee testing apparatus cab include a portion of the robot being positioned beneath a knee of a leg when the patient is situated in a supine position on the patient support.

In one example according to the teachings of the present invention, a robotic knee testing apparatus can include a robot configured to evaluate a knee of a patient, a patient support configured to support the patient thereon, and a positioning system adjustably connected to the patient support and movable relative to the patient support. The positioning system is movable to adjust a vertical position of the robot relative to the patient support.

In one example, the positioning system of the robotic knee testing apparatus can be movable to reposition the robot spaced from a distal edge of the patient support to allow the patient to pass between the distal edge of the patient support and the robot to get up onto the patient support.

In one example, the positioning system of the robotic knee testing apparatus can include a guide with at least one guide rod with a first end attached to a base of the positioning system and a second end slidably coupled to the patient support, the guide permitting the robot to move relative to the patient support in a horizontal direction.

In one example, the vertical position of the robot of the robotic knee testing apparatus can be adjustable to permit a knee of the patient to be in flexion in a range of 0 degrees to 90 degrees.

In one example, the positioning system of the robotic knee testing apparatus can further include a column lift with first and second column segments that are telescopically connected to one another to adjust the vertical position of the robot.

In one example, the positioning system of the robotic knee testing apparatus can further include a guide permitting the robot to move relative to the patient support in a horizontal direction, and a locking mechanism to lock the positioning system in a selected horizontal position so as to fix the horizontal position of the robot relative to the patient support.

In one example according to the teachings of the present invention, a knee examination method including the steps of positioning a patient adjacent a robotic knee testing apparatus having a patient support and a robot, moving a positioning system that supports the robot in a horizontal direction away from the patient support to create a gap between a distal edge of the patient support and the robot, situating the patient within the gap so that the patient can climb onto the patient support, and adjusting a horizontal position of the robot relative to the patient support using the positioning system to close the gap.

In one example, the knee examination method can further include the steps of repositioning the patient in a supine position on the patient support; and locating a knee stabilizer of the robot under a knee of the patient to support the knee.

In one example, the knee examination method can further include the steps of engaging a foot of the patient in a foot plate of the robot and can further adjust a vertical position of the robot relative to the patient support to place the knee of the patient in a desired degree of flexion.

In one example, the knee examination method steps can include the step of further adjusting results in flexion of the knee in a range of 0 degrees to 90 degrees.

### BRIEF DESCRIPTION OF THE DRAWINGS

Objects, features, and advantages of the present invention will become apparent upon reading the following description in conjunction with the drawing figures, in which:

FIG. 1 shows a perspective view of one example of a robotic knee testing or RKT apparatus according to the teachings of the present disclosure.

FIG. 2 shows an end view of the RKT apparatus when viewed from the right hand side in FIG. 1.

FIG. 3 shows an exploded view of a tibia positioning assembly of a robot of the RKT apparatus of FIG. 1.

FIG. 4 shows a robot of the RKT apparatus of FIG. 1 and depicts left and right legs of a patient positioned relative to the left and right leg portions of the robot.

FIG. 5 shows a right leg portion of the robot of FIG. 4 and depicts an X-Y-Z coordinate system defined by the right leg portion.

FIG. 6 shows an enlarged perspective view of part of the right leg portion of the robot of FIG. 1.

FIG. 7 shows a side view of the right leg portion of the robot of FIG. 5 and illustrates anterior-posterior motion about the X-axis of the tibia positioning assembly of the right leg portion of the robot.

FIG. 8 shows a top view of the robot of FIG. 4 and illustrates Varus-valgus motion about the Y-axis of the tibia positioning assembly of each of the left and right leg portions of the robot.

FIG. 9 shows an end view of the robot of FIG. 4 when viewed from the left-hand side in FIG. 1 and illustrates internal and external rotation about the Z-axis of each of the left and right leg portions of the robot.

FIG. 10 shows an environment view of a system utilizing the RKT apparatus of FIG. 1.

FIG. 11 shows a flow chart of one example of a set-up and knee laxity test method according to the teachings of the present disclosure.

FIG. 12 shows a flow chart depicting additional steps for each of the patient set-up and robot set-up steps of FIG. 11.

FIG. 13 shows a side view of the positioning system of FIG. 13 and in the extended position spaced from a table assembly of the RKT apparatus.

FIG. 14 shows a perspective view of one example of a positioning system of the RKT apparatus of FIG. 1 and constructed in accordance with the teachings of the present disclosure.

FIG. 15 shows a partial exploded view of the positioning system of FIG. 13.

FIG. 16 shows a perspective view of a locking mechanism of the positioning system of FIG. 13.

FIG. 17 shows a perspective view of the locking mechanism of FIG. 16 and in a locked state.

FIG. 18 shows a partial exploded view of a knee stabilizer and a thigh immobilizer of the RKT apparatus of FIG. 1.

### DETAILED DESCRIPTION OF THE DISCLOSURE

The disclosed RKT apparatus and positioning system are intended to aid a patient with ingress and egress relative to the RKT apparatus. The disclosed RKT apparatus and positioning system also permits testing of a patient's knee with the knee in a range of flexion angles. The disclosed RKT apparatus, positioning system, and method, in turn, are intended to aid or assist patients to receive testing. Further, the disclosed RKT apparatus, positioning system, and method significantly improve knee testing methods, thereby yielding improved test results. These and other objects, features, and advantages of the present disclosure will become apparent to those having ordinary skill in the art upon reading this disclosure.

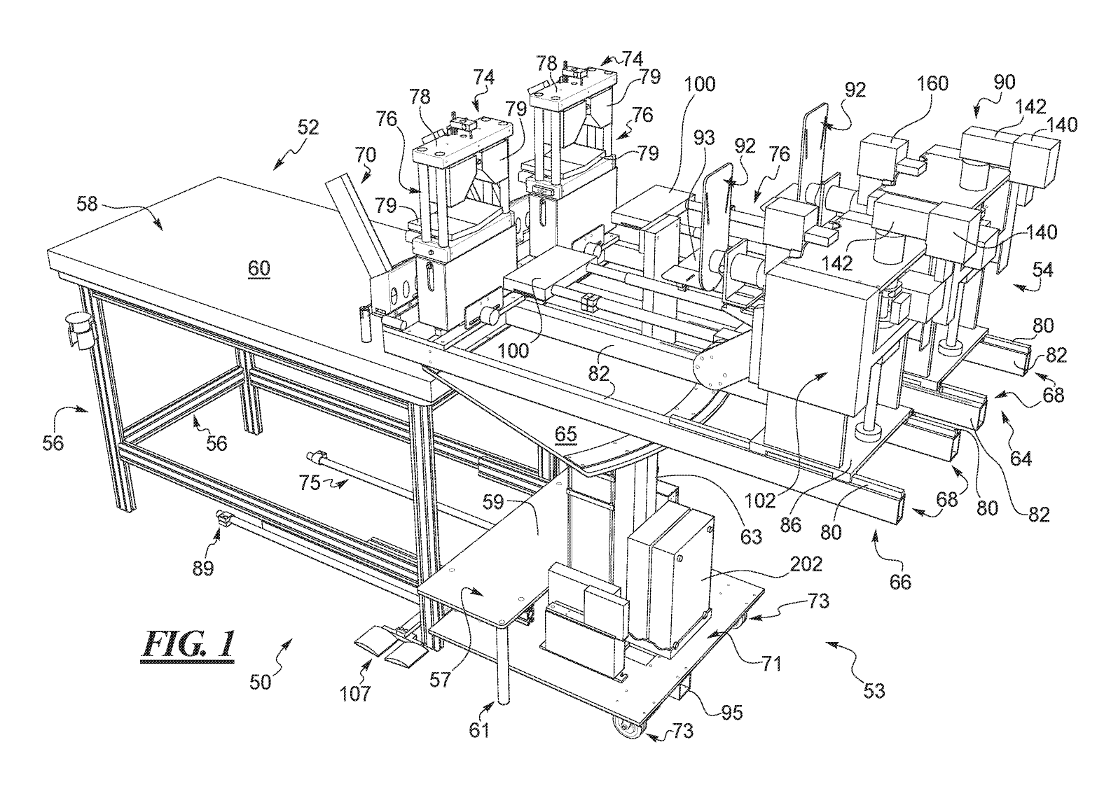

Turning now to the drawings, FIG. 1 shows one example of a RKT apparatus 50 that has been developed by the applicant and assignee of the present inventions that are disclosed and described herein. Specific details of the RKT apparatus 50 are more fully disclosed and described in the above-noted U.S. publication no. 2014/0081181 (“181”), which is owned by the applicant and assignee of the inventions disclosed herein. Specific details of the overall function and operation of the robotic portion of the RKT apparatus are described in the '181 publication and in the above-noted U.S. publication no. 2012/0046540, which is also owned by the applicant and assignee of the inventions disclosed herein. The entire content of both of the '181 and '540 publications are hereby incorporated herein by reference.

The RKT apparatus 50 of FIG. 1 generally has a patient support, i.e., a table assembly 52. The RKT apparatus 50 also has a robotic mechanism or limb manipulation device, identified for ease of description herein as a robot 54, positioned at one end or edge of the table assembly 52. The robot 54 is supported by a robot positioning system 53 that is configured so that the robot is movable relative to the table assembly 52. The table assembly 52 in this example has a supporting frame that is identified herein as a base 56 beneath a patient platform 58. The base 56 is configured to rest on a floor or surface and to support the patient platform 58 above the floor. The patient platform 58 can include a substantially rigid or sturdy panel (not shown) capable of holding and supporting a patient thereon. The panel can be affixed to or otherwise supported by the base 56. The panel of the patient platform 58 can underlie a padded surface 60, which can include a textile or fabric material that covers a cushion, padding, or the like (also not shown).

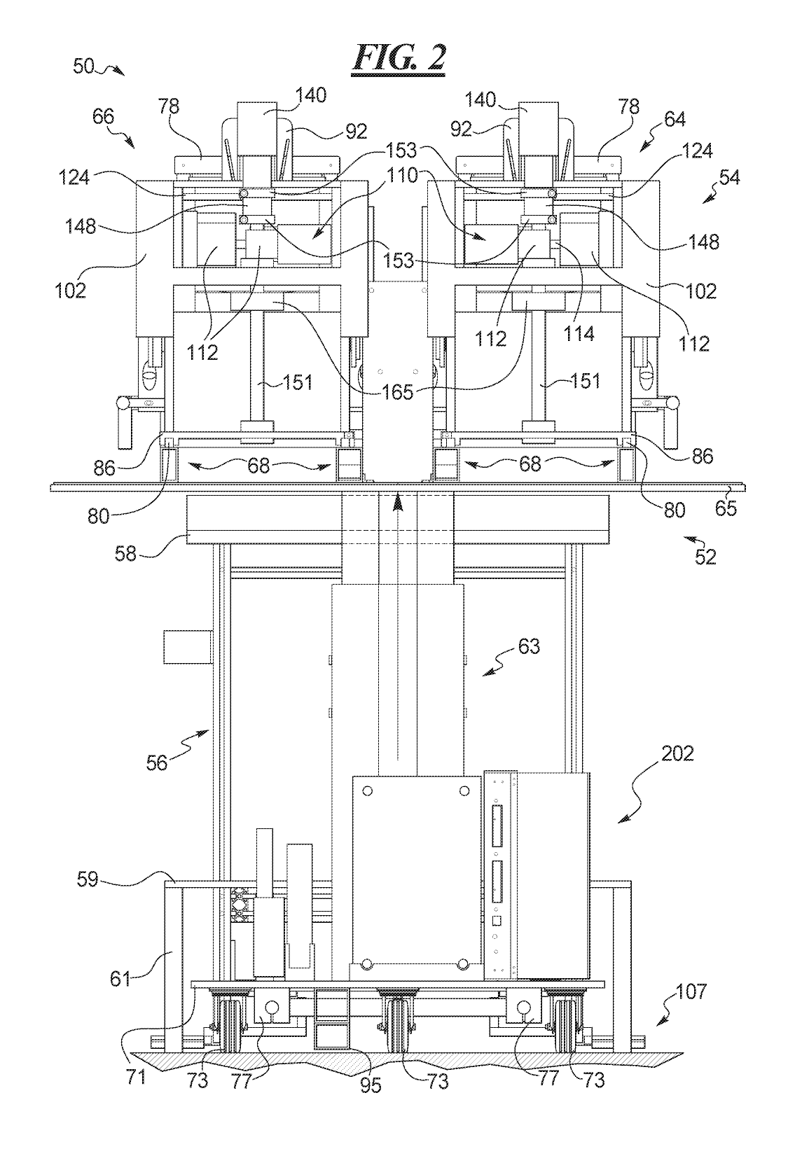

As shown in FIGS. 1 and 2, the patient support can include a step 57 positioned at the distal end of the table assembly 52 to assist a patient to step up onto the patient platform 58. The step 57 includes a tread 59 that can include a substantially rigid or sturdy panel capable of supporting a patient thereon while getting on and off of the table assembly 52. The tread 59 can be supported by a step base 61 that is configured to rest on a floor or surface and to support the tread above the floor. The tread 59 may also be supported by the base 56. The step base 61 and/or tread 59 can also be affixed to or separate from the base 56. Further, the step base 61 and/or tread 59 may be formed or provided as an integral part of the table assembly 52.

As will be evident to those having ordinary skill in the art, the configuration and construction of the table assembly 52 and step 57 can vary considerably from the example disclosed, illustrated, and briefly described herein. The base 56 and/or the patient platform 58 and step base 61 and/or tread 59 can each be altered in configuration, size, shape, orientation, height, construction, materials, and the like. The base 56 and step 57 can include multiple legs and frame elements that are assembled or connected to one another, as in the illustrated example. Alternatively, the base 56 and/or step 57 can be formed as one unitary support element. The patient platform 58 and/or step base 61 can also be formed of multiple components and can be fastened to or otherwise attached to the base. Alternatively, the patient platform 58 and/or step base 61 can be an integral, one-piece fabricated structure and can be fabricated as part of the base or attached thereto. The patient support need not be a table, but instead can be a chair, a suspension system, or other suitable structure that is capable of properly positioning and retaining a patient relative to the robot 54 for testing and examination. The table assembly 52 and/or step 57 can further include additional features, though not disclosed or described herein, that may be used to assist in the patient sitting on the patient platform, to assist in positioning a patient on the patient platform, to assist in maintaining a patient's position on the platform 58, or to otherwise enhance patient comfort or improve performance of the table assembly, the RKT apparatus, or both.

With reference to FIGS. 1 and 2, the positioning system 53 of the RKT apparatus 50 can be configured to allow moving the robot 54 relative to the table assembly 52. The positioning system 53 is adjustably connected to the table assembly 52 in this example. The positioning system 53 has a column lift 63 that can raise and lower the robot 54 as well. Details of the positioning system 53 are described in more detail below. In this example, the positioning system 53 is configured to further assist a patient in getting onto the patient platform 58, as well as to aid in positioning the patient for testing.

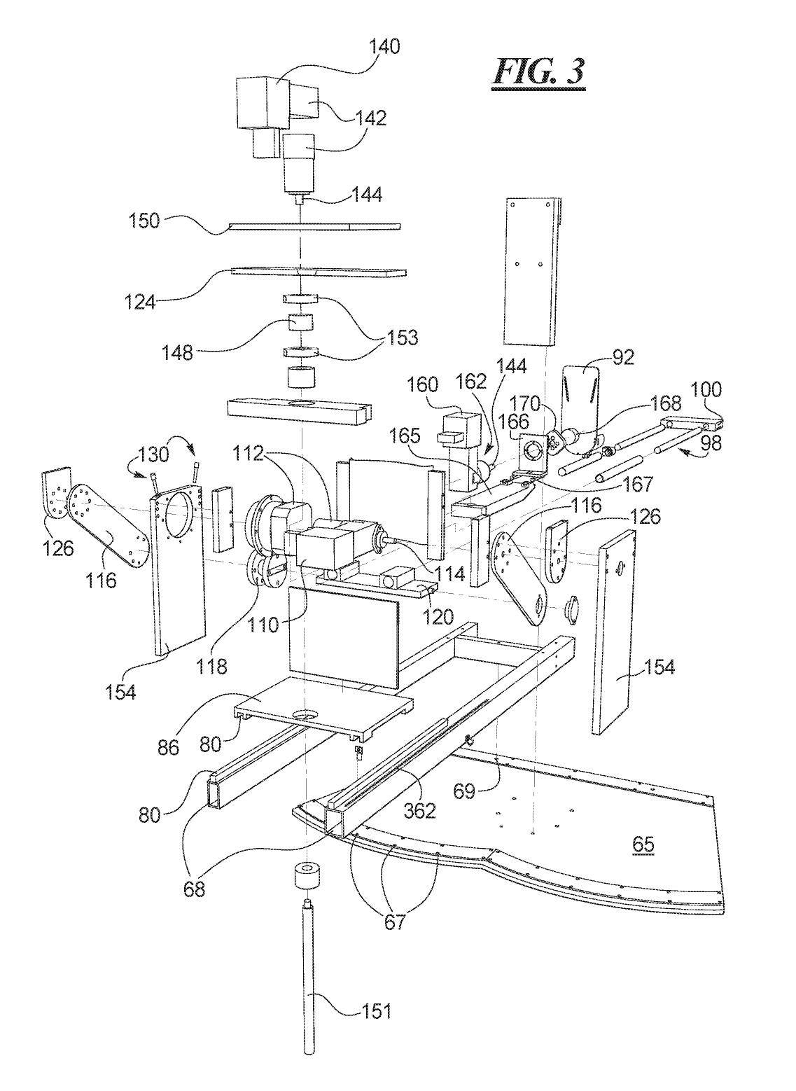

In the disclosed example and with reference to FIGS. 2 and 3, the robot 54 has a left leg testing and evaluation mechanism and a right leg testing and evaluation mechanism, each mechanism respectively identified herein as a left leg portion 64 and a right leg portion 66 of the robot. The left and right leg portions 64, 66 have substantially the same construction, and may be essentially identical, if desired, and each is constructed to support and evaluate a left leg and right leg, respectively, of a patient. Therefore, like reference numerals are used herein to identify common parts of each of the two leg portions 64, 66 that have the same construction. The left and right leg portions 64, 66 each have a sub-frame 68 that, in this example, is supported directly or indirectly by the robot positioning system 53. Each sub-frame 68 supports the components and parts of the corresponding left and right leg portions 64, 66. For ease of description, the right leg portion 66 of the robot 54 is described in more detail below with the understanding that the left leg portion 64 has or may have the same overall construction. Differences between the two leg portions are identified herein, if and as needed. It is possible that an RKT apparatus is provided that has only one leg portion for evaluating only one leg of a patient at a time. However, in the disclosed example, the RKT apparatus 50 has left and right leg portions 64, 66.

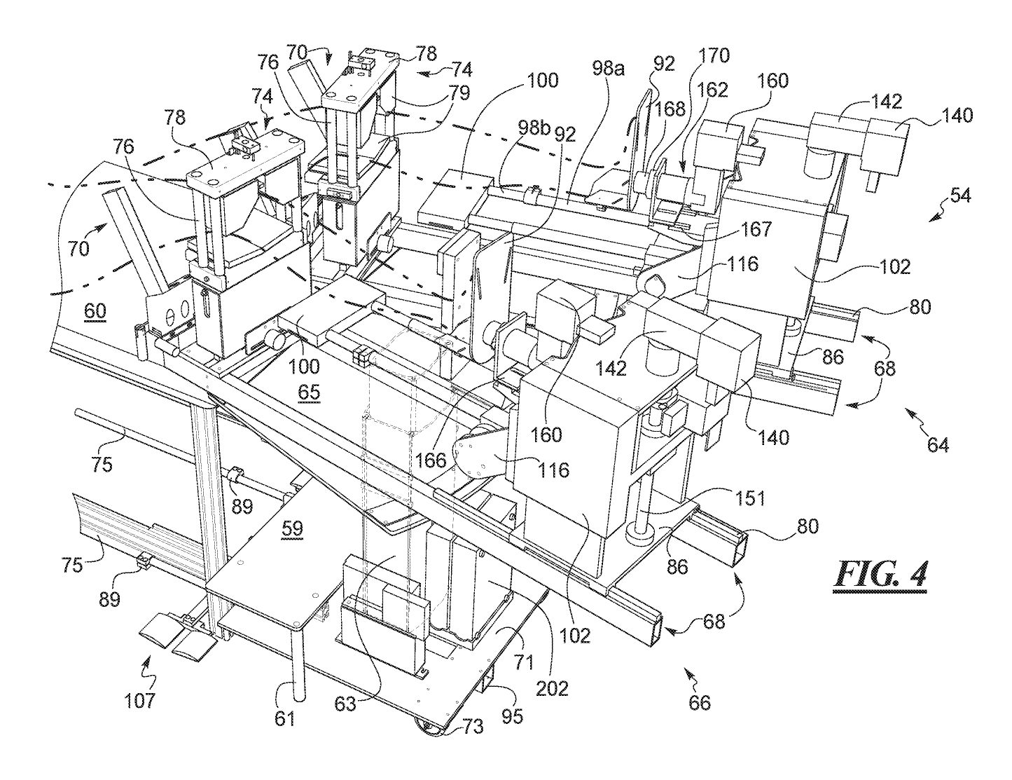

As depicted in FIGS. 3-5, the right leg portion 66 has a thigh clamp or immobilizer 70 positioned closest to the table assembly 52. The thigh immobilizer 70 can be mounted to the robot support 65 or the sub-frame 68, or can be otherwise mounted to a portion of the RKT apparatus 50 in a manner suitable for use as described below. The thigh immobilizer 70 can be constructed so as to be positionally adjustable to accommodate a wide range of patients of different size. The thigh immobilizer 70 should be positioned or positionable to contact a portion of a patient's upper leg or thigh above the knee, as depicted in FIG. 4. The thigh immobilizer 70 has a pair of femur clamping elements 72 that are side-to-side adjustable to clamp onto and hold a patient's thigh.

The thigh immobilizer 70 in this example has a pair of femur clamping elements 72, i.e., medial and lateral clamping elements that are spaced apart and width-wise adjustable relative to one another. Though not shown herein, the clamping elements 72 can include a pad or pads on the thigh facing surfaces, if desired, to provide a degree of comfort for a patient. The femur clamping elements 72 can be side-to-side adjusted in order to clamp or otherwise securely hold a patient's right femur and thigh in a substantially fixed side-to-side position during testing, evaluation, or treatment, as described below. The configuration and construction of the thigh immobilizer 70 can vary considerably from the example shown herein. The clamping elements 72 can be replaced by other suitable securing or clamping devices or elements and the mechanisms to adjust and secure the thigh immobilizer 70 can also vary.

In the example shown in FIGS. 3-5, the right leg portion 66 also has a knee stabilizer 74 positioned adjacent the thigh immobilizer 70. The knee stabilizer 74 can also be mounted to the robot support 65 or the sub-frame 68, or can be otherwise mounted to a portion of the RKT apparatus 50 in a manner suitable for use as described below. The knee stabilizer 74 can optionally also be constructed so as to be positionally adjustable to accommodate a wide range of patients of different size. The knee stabilizer 74 should be positioned or positionable to contact the knee or patella at the lower end of a patient's femur and thigh, as depicted in FIG. 4.

The knee stabilizer 74 acts as a knee or patellar clamp and can include a framework 76 arranged to surround and clamp onto a patient's joint or knee. The knee stabilizer 74 in this example has a pair of patellar clamping elements 78 that are vertically spaced apart and adjustable relative to one another along the framework 76. The patellar clamping elements 78 can be vertically adjusted in order to clamp or otherwise securely hold the lower end of a patient's right femur and patella in a substantially fixed vertical position during testing, evaluation, or treatment, as described below. If the knee stabilizer 74 is positionally adjustable, it should be capable of being secured in a fixed selected position, once properly adjusted for a given patient, relative to the table assembly 52 and/or robot 54 during testing. The configuration and construction of the knee stabilizer 74 can vary considerably from the example shown herein. The patellar clamping elements 78 can be replaced by other suitable securing or clamping devices or elements and the mechanisms to adjust and secure the knee stabilizer 74 can also vary.

The knee stabilizer 74 can include a plurality of substantially rigid and/or resilient pads 79, such as on the upper and lower patellar clamping elements 78. The pads 79 can be configured and arranged to lie adjacent the patient's knee, preventing the framework 76 and the patellar clamping elements 78 from directly contacting the patient's knee. The pads 79 can be solid, hollow, pressurized, hydraulically filled, pneumatically filled, or the like and can be rubber, foam, or otherwise formed of suitable materials. In one example, the pad or pads 79 on the upper patellar clamping element 78 can be configured to define a V-shape within the framework 76. The patient's leg can then be captured within the V-shape as the upper and lower patellar clamping elements 78 are drawn toward one another to capture and hold still the patient's leg during a procedure.

The thigh immobilizer 70 and/or the knee stabilizer 74 may be mechanically adjustable to manually fit and accommodate different sized patients. In one alternative, the thigh immobilizer 70 and/or the knee stabilizer 74 may be electrically operable to adjust the femur clamping elements 72, the patellar clamping elements 78, respectively, or both. In another alternative example, the femur clamping elements 72 and/or the patellar clamping elements 78 may be pneumatically or hydraulically operable to adjust the thigh immobilizers 70 and knee stabilizers 74. In yet another alternative, the thigh immobilizer 70, the knee stabilizer 74, or both, may include two or more such systems or mechanisms for adjusting the respective clamping elements.

The thigh immobilizer 70 and/or femur clamping elements 72 and the knee stabilizer 74 and/or framework 76 and patellar clamping elements 78 can be formed of metal, plastic, or other suitable materials. The thigh and knee stabilizers 70 and 74 can vary in shape, configuration and construction, as desired. The thigh immobilizers 70 and knee stabilizers 74, in combination, are intended to secure a patient's leg in order to hold the femur and patella in a vertically (knee stabilizer) and side-to-side (thigh stabilizer) fixed position during a test, evaluation, or treatment cycle. Features and aspects of the disclosed thigh immobilizers 70 and knee stabilizers 74 can vary considerably while accomplishing this objective.

In this example as shown in FIGS. 3-5, the sub-frame 68 is configured to define or carry one or more slide tracks 80. The track or tracks 80 can be carried on the free end of the sub-frame 68 that is distal or spaced from the table assembly 52. The sub-frame 68 is formed having a plurality of rails 82 that extend lengthwise. The tracks 80 can be formed as an integrated part of the rails 82 or other sub-frame components or, as in this example, can be separately mounted to or supported by the rails. One or more trucks or carriages, hereinafter a sled assembly 86 is mounted on or supported by the sub-frame 68 and is slidable along the tracks 80.

As depicted in FIG. 3, portions of the robot 54 are supported on the sled assembly 86 via support brackets 154. Support brackets 154 may be coupled to the sled assembly 86. The support brackets may be rectangular plates connected together by a cross-plate 120. Vertical supports may rest upon the cross-plate and support a pivot plate 150.

As depicted in FIGS. 2-4, the right leg portion 66 further includes a tibia positioning assembly 90 that is mounted on the sub-frame 68. In this example, the tibia positioning assembly 90, or at least a portion of the assembly, is carried on the sled assembly 86. Thus, the tibia positioning assembly 90, or at least a portion thereof, is slidable lengthwise along the tracks 80 of the sub-frame 68 on the sled assembly 86, and thus is movable relative to the table assembly 52 and/or to the thigh and knee stabilizers 70 and 74.

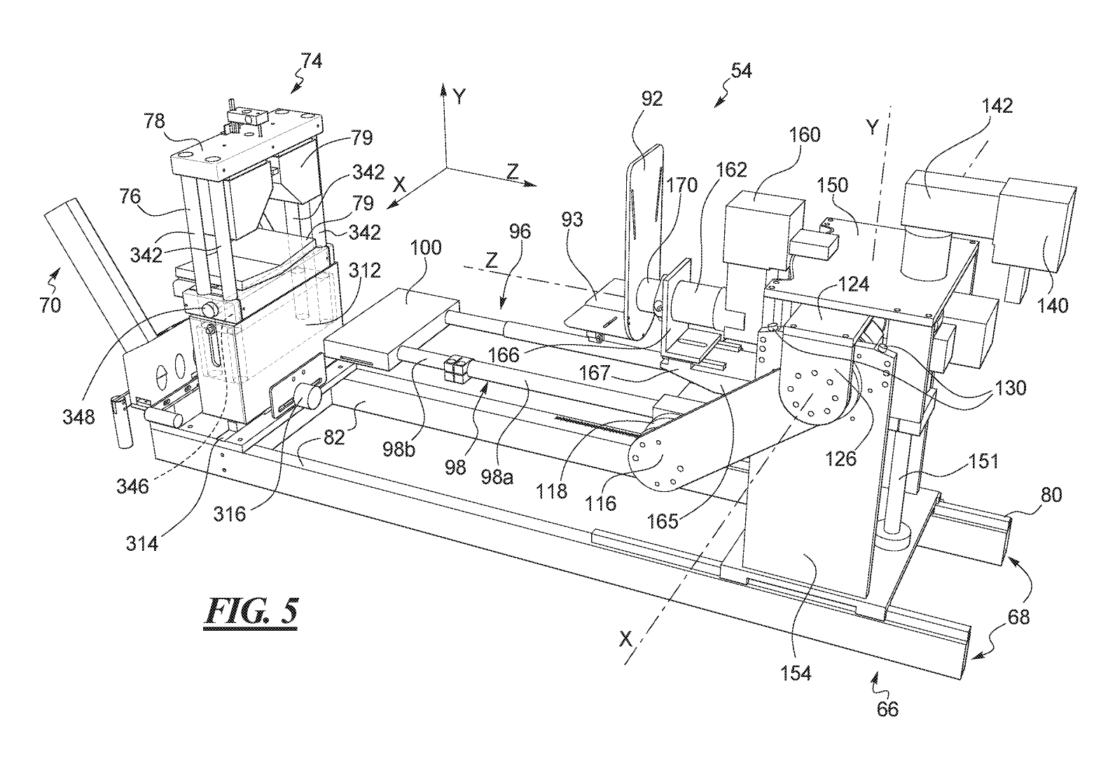

In general, the tibia positioning assembly 90 has a foot holder, i.e., a foot plate 92 in this example with a heel stop 93 at the bottom edge of the foot plate that faces upward and has a contact surface 94 that faces toward the thigh and knee stabilizers 70 and 74. The tibia positioning assembly 90 also has a tibia rod device 96 with one or more rods 98 and a calf plate 100 at or near a distal end of the tibia rod device. The one or more rods 98 can be lengthwise adjustable. In this example as shown in FIGS. 5-8, the tibia rod device 96 has two tibia rods 98, each of which has two telescoping segments including a fixed segment 98a and a slidable segment 98b that permit length adjustment of the rods 98. Though not shown or described herein, the rods 98 may include a locking mechanism or a suitable type, such as holes and set screws, VALCO ball devices, or the like on one or both of the segments 98a, 98b, that can lock the adjusted rods 98 at a selected length. The telescoping segments permit adjustable positioning of the calf plate 100 relative to the foot plate 92 to accommodate different sized patients. During use, the calf plate 100 lies under and contacts a patient's calf below the knee and the foot plate 92 bears against the sole of the patient's foot. The foot plate 92 can be configured to physically constrain and hold the foot of a patient against the contact surface 94. In one example, though not shown herein, the foot plate 92 can employ one or more straps that secure the patient's heel against the heel stop 93 and the sole of their foot to the foot plate 92. Likewise, the calf plate 100 can be configured to physically constrain the patient's leg to the calf plate, as described below for certain tests, or can merely lie against and under the patient's calf while not being otherwise secured to the leg for other tests.

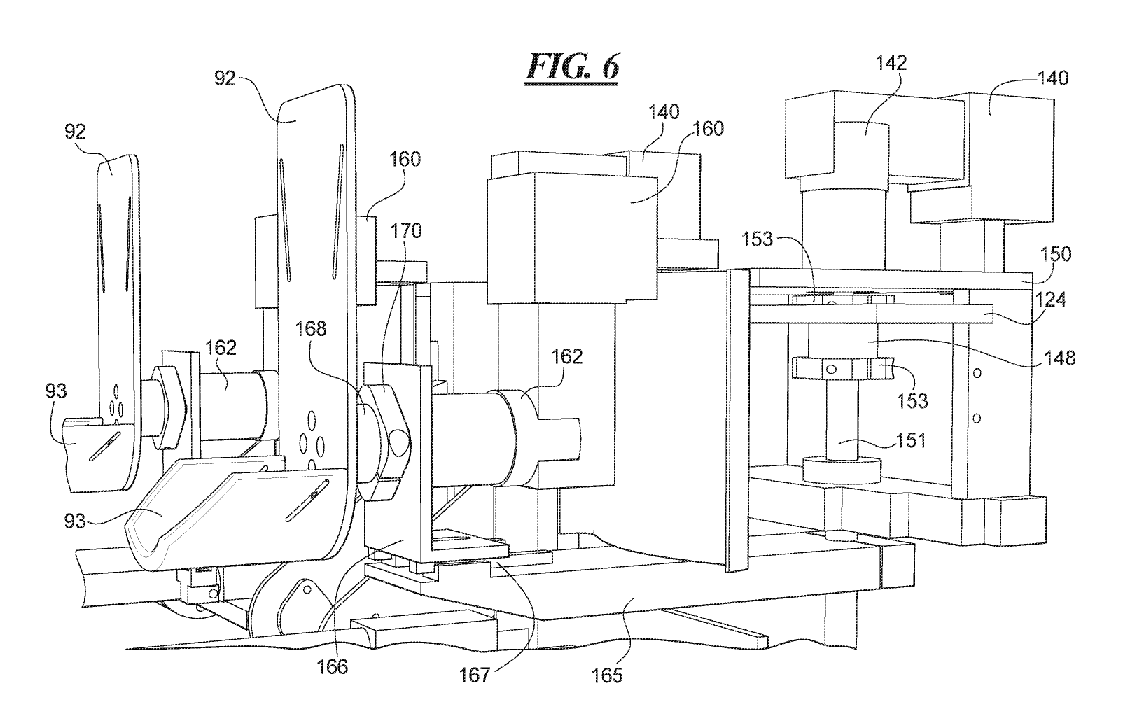

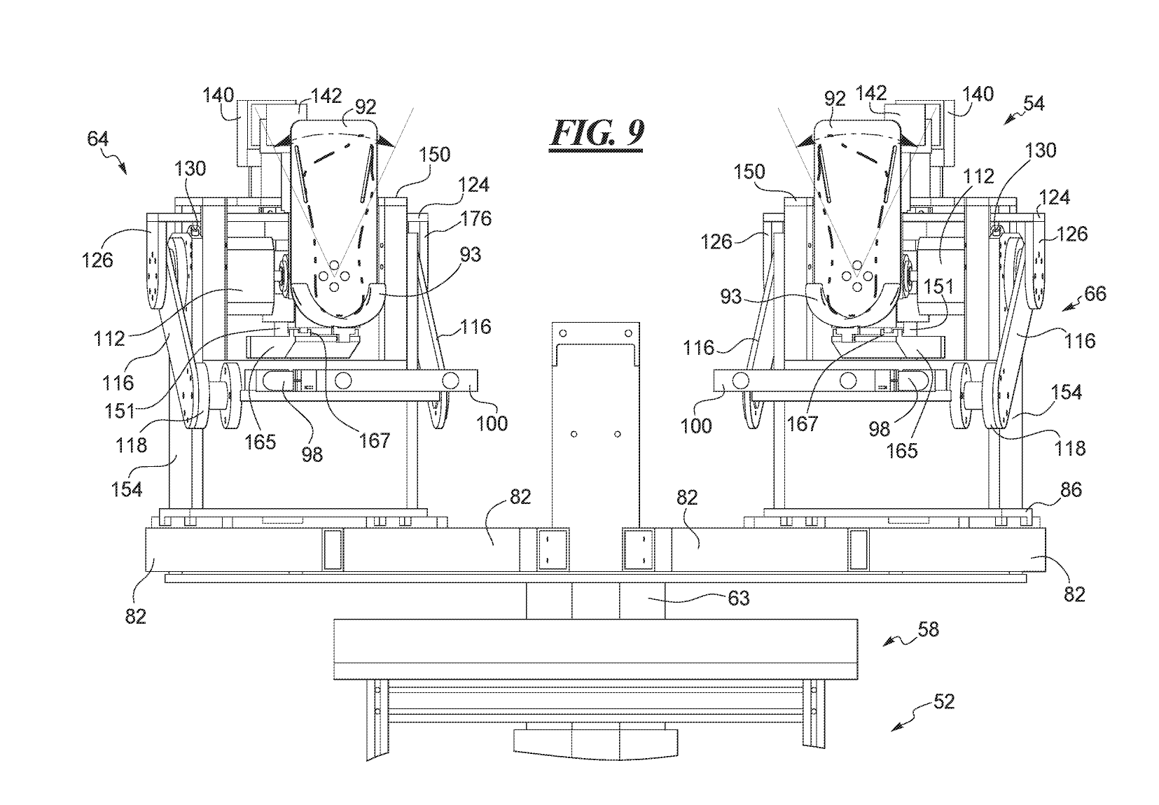

With reference to FIGS. 6-8, the tibia positioning assembly 90 has a drive system with a number of drive components configured to impart specific and controllable movements to the lower leg of a patient. In this example, a number of the drive system components are housed within a shell or housing 102. In other examples, the drive system components may be exposed and the shell eliminated. The drive system in this example generally has a first drive, i.e., an X-axis drive 104 as identified herein, which is oriented to define and provide rotation about a first axis, i.e., an X-axis as identified herein, which in this example lies generally across the tibia positioning assembly 90. The drive system also has a second drive, i.e., a Y-axis drive 106 as identified herein, which is oriented to define and provide rotation about a second axis, i.e., a Y-axis as identified herein, which in this example lies generally vertically through the tibia positioning assembly 90, though not quite intersecting the X-axis, as described below. The drive system further has a third drive, i.e., a Z-axis drive 108 as identified herein, which is oriented to define and provide rotation about a third axis, i.e., a Z-axis as identified herein (see FIG. 9), which in this example lies lengthwise along the tibia positioning assembly 90. The three axes define a coordinate system and this coordinate system is identified as an X-Y-Z coordinate system for the right leg portion 66 of the robot 54 in this example. The robot 54 will also have a similar X-Y-Z coordinate system specific to the left leg portion 64, but independent of the coordinate system for the right leg portion 66.

In other examples, the RKT apparatus may be configured to test only one or two of anterior-posterior motion, Varus-valgus motion, or tibial rotation, instead of all three tests. In such cases, the drive system may include only one or two of the X-axis, Y-axis, or Z-axis drives instead of all three drives. The methods and procedures described herein may be modified to accommodate such robots that have fewer than all three drives. In other examples, the X-Y-Z axes of the aforementioned coordinate systems may all intersect with one another and may all be orthogonal to one another. In still other examples, none or only two of the axes may intersect and/or none or only two of the axes may be orthogonal to one another.

As shown in FIG. 3, the X-axis drive 104 can include a first motor, such as an electric motor 110, a gearbox 112, and an output shaft 114 that is driven by the motor and gearbox. The opposite ends of the output shaft 114 in this example are fixedly coupled to the upper ends of respective drive links 116 on opposite sides of the housing 102. Thus, as the output shaft 114 is rotated by the motor 110 and gearbox 112, the drive links 116 are also rotated about the X-axis. The drive links 116 in this example are oriented downward and forward from the X-axis. The lower end of one of the drive links 116 is coupled or fixed to an X-axis torque transducer 118. The torque transducer 118 is also coupled or fixed to one end of a cross-plate 120. The lower end of the other drive link 116 is fixed to the opposite end of the cross-plate 120. The cross-plate 120 is coupled to and extends sideways across the right leg portion 66 forward of the X-axis between the drive links 116. In this example, the fixed segments 98a of the tibia rods 98 are fixedly mounted to and extend forward toward the knee and thigh stabilizers 70, 74 from the cross-plate 120, as shown in FIGS. 4 and 5.

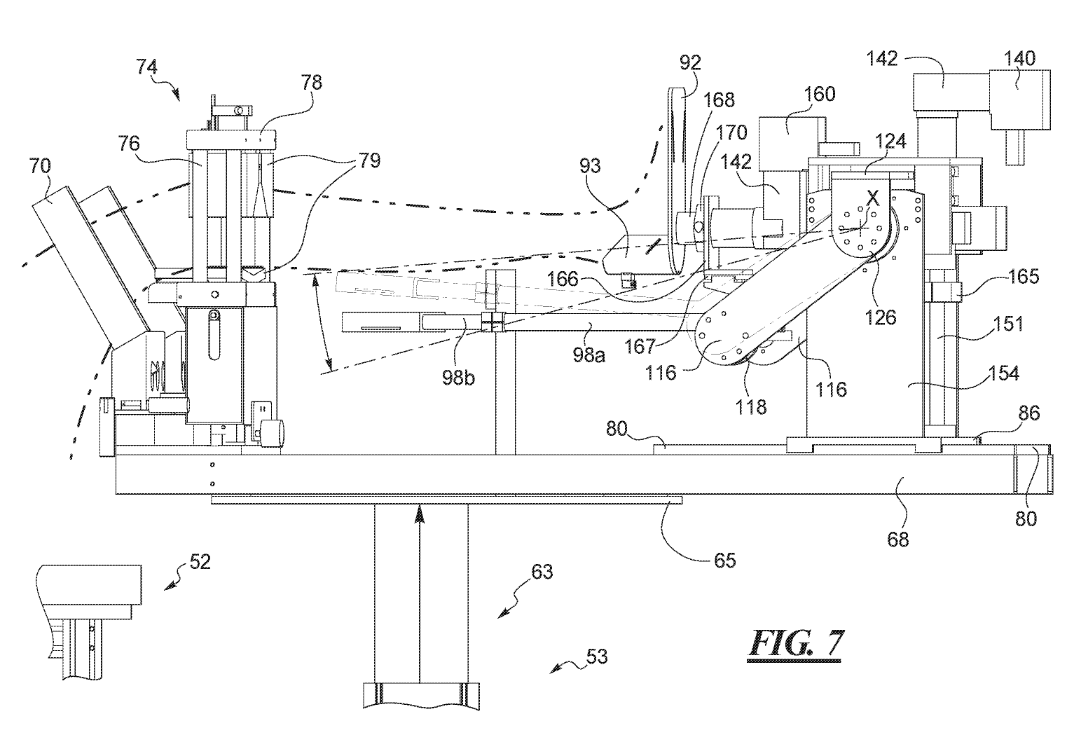

With reference to FIG. 7, the X-axis drive 104 is configured to conduct an anterior-posterior or A-P test on a patient's knee. Position sensors can be applied to appropriate locations on the right leg of the patient. The X-axis drive 104 imparts force about the X-axis to initiate anterior-posterior motion in the tibia part of the knee joint relative to the fixed femur part of the knee joint of the patient, as shown in FIG. 7. The motor 110 can reversibly rotate the output shaft 114 through an arc about the X-axis whereby the upper ends of the drive links 116 are rotated through the same arc. This in turn moves, i.e., raises or lowers the lower ends of the drive links 116, which in turn raises or lowers the cross-plate 120 and the fixed segments 98a of the tibia rods 98. Movement of the fixed segments 98a of the tibia rods 98 raises or lowers the slider segments 98b and thus the calf plate 100 carried on the tibia rods 98. The X-axis torque transducer 118 measures the applied torque at the cross-plate 120 caused by the load applied at the calf plate 100 as the calf plate pushes up on the patient's tibia or the tibia rods 98 pull down on the patient's tibia. Motion and load data can be collected by a processor from the sensors relative to the motion in the patient's leg and from the X-axis torque transducer 118 relative to the torque or applied force.

The motor 110 and/or gearbox 112 can be designed to produce a limited range of travel, which may be substantially less than 360 degrees of rotations, in the output shaft 114. In addition or in the alternative, the X-axis drive 104 can also be designed to incorporate a mechanical travel limiter, if desired. In one example as shown in FIGS. 3, 5, and 7, a yolk assembly 122 can be provided as part of the X-axis drive 104. The yolk assembly 122 has a top plate 124 extending over a top of the housing 102. The yolk assembly 122 also has a pair of side plates 126 extending down from the top plate 124. The side plates 126 can be affixed to the upper ends of the drive links or otherwise to the drive shaft 114 of the motor 110, so that the yolk assembly 122 also rotates with the drive shaft 114. Two stops 130, i.e., fore and aft travel stops protrude upward from the support bracket 154. The stops 130 are positioned and circumferentially spaced apart relative to the X-axis. The top plate 124 of the yoke assembly 122 is captured between the two stops and hits one of the stops to limit travel of the yoke assembly in either rotation direction. The radius of the side plates 126 and spacing of the stops 130 can thus limit rotational travel of the output shaft 114 to a specific arc, which mechanically limits the upward and downward travel of the tibia rods 98.

The above-described anterior-posterior movement components of the tibia positioning assembly 90 can vary considerably from the example shown and described herein. The yoke assembly 122 and stop bracket 128 can be eliminated or can take on different positions, configurations, and constructions. Instead, another mechanical stop mechanism can be employed. Likewise, the configuration and construction of the drive links 116, cross-plate 120, tibia rods 98, and calf plate 100 can also be varied. The mechanisms or devices that are used to secure a patient's leg to the tibia rods 98 and to the foot plate 92, if and when needed for testing, can also vary.

As shown in FIGS. 3-6, the Y-axis drive 106 can include a second motor, which can also be an electric motor 140, a gearbox 142, and an output shaft 144 that is driven by the motor and gearbox. The gearbox 142 and motor 140 are fixed to a pivot plate 150 above the X-axis drive 104. The output shaft 144 is connected to a vertical drive shaft 151 via a coupler 153. The drive shaft 151 can be pivotably coupled to the sled assembly 86. The Y-axis drive pivots around the drive shaft 151. A Y-axis torque transducer 148 is fixed to the output shaft 144 for rotation therewith via a coupler 153 and to the drive shaft 151 via another coupler 153.

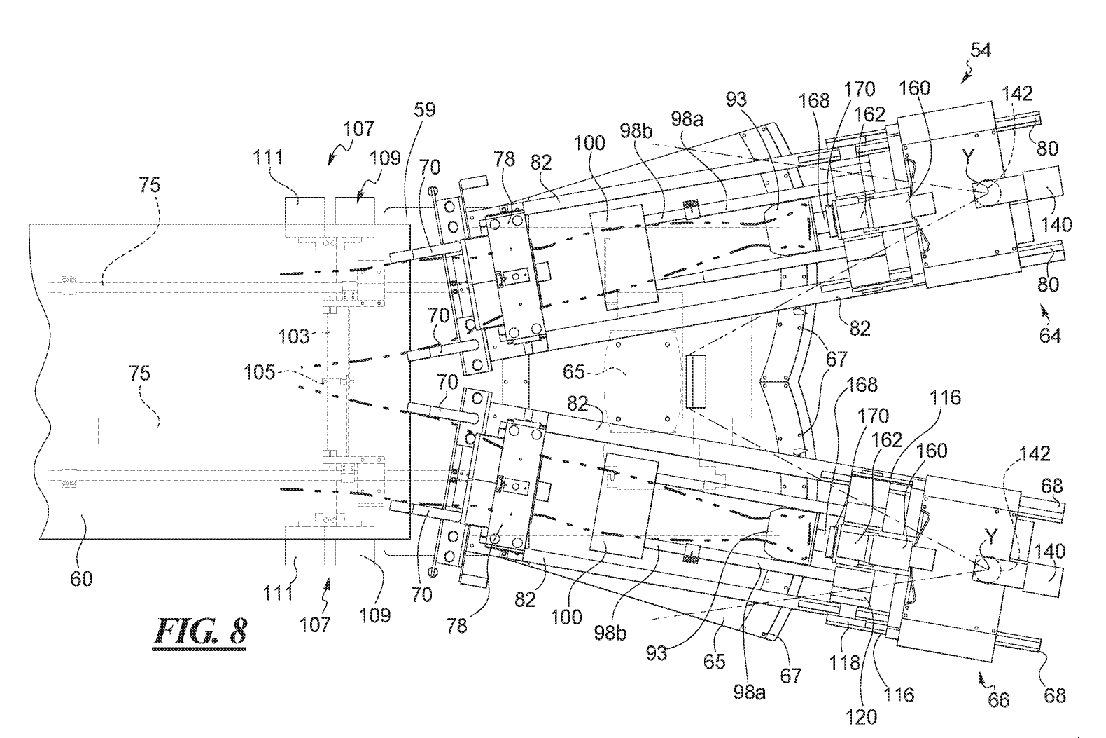

As represented in FIG. 8, the Y-axis drive 106 is configured to conduct a Varus-valgus or V-V test on a patient's knee. Position sensors can be applied to appropriate locations on the right leg of the patient. The Y-axis drive 106 imparts force about the Y-axis to initiate Varus-valgus motion in the tibia part of the knee joint relative to the fixed femur part of the knee joint of the patient, as shown in FIG. 8. The motor 140 can reversibly rotate the output shaft 144 through an arc about the Y-axis. The Y-axis torque transducer 148 measures the applied torque at the output shaft 144 caused by the load applied at the foot plate 92 or along the foot plate support 165 as the foot plate 92 pushes the patient's tibia medially or laterally relative to the femur. Motion and load data can be collected by a processor from the sensors relative to the motion in the patient's leg and from the Y-axis torque transducer 148 relative to the torque or applied forces.

The motor 140 and/or gearbox 142 can be designed to produce a limited range of travel, which may be substantially less than 360 degrees of rotations, in the output shaft 144. In addition or in the alternative, the Y-axis drive 106 components can also be designed to incorporate a mechanical travel limiter, if desired, though not shown or described herein.

The above-described Varus-valgus movement components of the tibia positioning assembly 90 can also vary considerably from the example shown and described herein. The sled assembly 86, pivot plate 150, and support brackets 154 can be eliminated or can take on different positions, configurations, and constructions. The mechanisms or devices that are used to secure a patient's leg to the tibia rods 98 and to the foot plate 92, if and when needed for testing, can also vary.

As shown in FIGS. 3, 5, and 9, the Z-axis drive 108 can include a third motor, which can also be an electric motor 160, a gearbox 162, and an output shaft 164 that is driven by the motor and gearbox. The gearbox 162 and motor 160 are fixed to a motor mounting bracket 166 that is attached to a foot plate support 165 through a slide bearing 167. The foot plate support 165 can be attached to the Y-axis drive shaft 151 in a key and keyway configuration (not shown). This assembly configuration results in the entire Z-axis drive and corresponding foot plate 92 pivoting about the Y-axis when conducting a Varus-valgus or V-V test on a patient's knee. A Z-axis torque transducer 168 is fixed to the output shaft 164 by an adaptor 170 for rotation therewith. The foot plate 92 is secured to the torque transducer 168 for rotation therewith. Thus, as the output shaft 164 is reversibly rotated by the motor 160 and gearbox 162 about the Z-axis. As shown in FIGS. 8 and 9, the foot plate 92 will all rotate about the Z-axis.

As represented in FIGS. 5, 6, and 9, the Z-axis drive 108 is configured to conduct an internal and external rotation or simply a tibia rotation test on a patient's knee. Position sensors can be applied to appropriate locations on the right leg of the patient. The Z-axis drive 108 imparts force about the Z-axis to initiate rotation motion in the tibia part of the knee joint relative to the fixed femur part of the knee joint of the patient, as shown in FIG. 9. The motor 160 can reversibly rotate the output shaft 164 through an arc about the Z-axis whereby the adapter 170 and torque transducer 168 are rotated through the same arc. This in turn moves, i.e., rotates the foot plate 92 about the Z-axis. Movement of the foot plate 92 in this manner rotates the patient's lower leg internally and externally relative to the femur. The Z-axis torque transducer 168 measures the applied torque at the output shaft 164 caused by the load applied at the foot plate 92 as the foot plate rotates the patient's tibia or lower leg internally and externally relative to the femur. Motion and load data can be collected by a processor from the sensors relative to the motion in the patient's leg and from the Z-axis torque transducer 168 relative to the torque or applied forces.

The motor 160 and/or gearbox 162 can be designed to produce a limited range of travel, which may be substantially less than 360 degrees of rotations, in the output shaft 164. In addition or in the alternative, the Z-axis drive 108 components can also be designed to incorporate a mechanical travel limiter, if desired. A simple mechanical stop can be positioned to stop movement of the foot plate 92 in either rotation direction, if desired. Such a stop can be the tibia rods 98 or something mounted thereto. Alternatively, such a stop can be applied to the motor mounting bracket 166 or the like.

The above-described rotation movement components of the tibia positioning assembly 90 can also vary considerably from the example shown and described herein. The foot plate 92 and motor mounting bracket 166 can be eliminated or can take on different positions, configurations, and constructions. The mechanisms or devices that are used to secure a patient's leg to the foot plate 92, if and when needed for testing, can also vary.

The above described motors, gearboxes, and output shafts can also vary within the scope of the disclosure. The motors can be servo-motors or other types of motors suitable for precise motion and torque control and for the loads to which the motors will be exposed during such limb testing and evaluation. Any of the first, second, or thirds, i.e., the X-, Y-, or Z-axis drives with respect to the motors and gearboxes can be structurally configured substantially the same relative to one another, with the only substantive difference being the relative axis of rotation about which each is oriented. Alternatively, each drive can incorporate a motor and/or gearbox that is different than one or both of the others as well. The torque transducers can be selected in order to provide torque readings as known in the art relating to each of the three drives. In other examples, one or more of the torque transducers may be replaced with other torque or load sensors or load sensing means. For example, motor current may be measured to determine the torque or load on the motor output shaft during use. Any suitable means for modeling torque may be used. The torque readings can be calibrated and calculated as needed to correspond to known torque or force values imparted to a patient's limb(s). Movement of the patient's body parts may be detected by non-invasive systems, as noted above, that utilize sensors or markers that are attached to the skin, including but not limited to vision, optoelectronic, ultrasonic, and electromagnetic motion analysis systems.

In use, a patient lies on the padded surface 60 of the patient platform 58 on the table assembly 52 as shown in FIG. 4. The patient's knees are positioned to engage the knee stabilizers 74, their thighs are positioned to engage the thigh stabilizers 70, their feet are positioned to engage the foot plates 92, and their calves are positioned to engage the tibia rods 98. The patient can then be secured to the foot plates, to the knee stabilizers, and to the thigh stabilizers for testing and evaluation. The patient's calves or tibias can also be secured to the tibia rods 98, as needed for specific testing. Movement of the lower leg of the patient may be detected by non-invasive systems utilizing sensors or markers that are attached to the skin, including but not limited too optoelectronic, ultrasonic, and electromagnetic motion analysis systems. In one example, the RKT apparatus can be configured so that the patient's knees are flexed to about 30 degrees between the femur and the tibia. However, the tests or evaluations may also include the additional capability to flex the knee from 0 to 90 degrees to allow for similar tests (such as the examples above) done for different degrees of knee flexion. The RKT apparatus allows for testing of a patient's knees at multiple angles via horizontal and vertical adjustments.

Any one of the X-, Y-, and Z-drives can be decoupled from any of the other two. In the disclosed example, each of the three drive assemblies may be operable with one or more of the other at the same time or can be decoupled from each of the other two and be operable independent of the other two. In other examples, two or more, and perhaps all three of the drives can be mutually coupled relative to one another such that movements are substantially simultaneously imposed upon the patient's legs during use of the RKT apparatus.

The aforementioned sensors can be provided on the legs of a patient, in the power lines of the RKT apparatus, and/or on the X-, Y-, and Z-drives to obtain desired position or location data as the lower leg is moved during testing and evaluation. The degree of movement of the patient's legs in the A-P test, the V-V test, and/or the rotation test can be measured by detecting the movements of the parts of the apparatus, the rotation of the drives, and/or the actual movements of the patient's legs. The torque encountered during each test and over the range of motion applied during each such movement may also be measured, suitably calibrated to the limb movement, and recorded.

As noted above, even testing and evaluation of knee joints using the RKT apparatus 50 can be inconsistent from patient to patient, from doctor to doctor, and from test procedure to test procedure by the same doctors and/or on the same patients. Such inconsistency is created at least in part because each stage or step of the setup and testing procedures can introduce error into the data. The cumulative error can become quite substantial and thus significantly affect the accuracy of the test results. As disclosed herein, important stages or steps for each test are patient set-up and robot set-up. According to the teachings of the present disclosure, providing a consistent method or procedure to get a patient set-up in the RKT apparatus 50 has been determined to aid in producing more consistent test results and reducing error in the data. Further, according to the teachings of the present disclosure, providing a consistent method or procedure to set up or initialize the robot 54 of the RKT apparatus 50 prior to testing a given patient has also been determined to aid in producing more consistent test results and reducing error in the data.

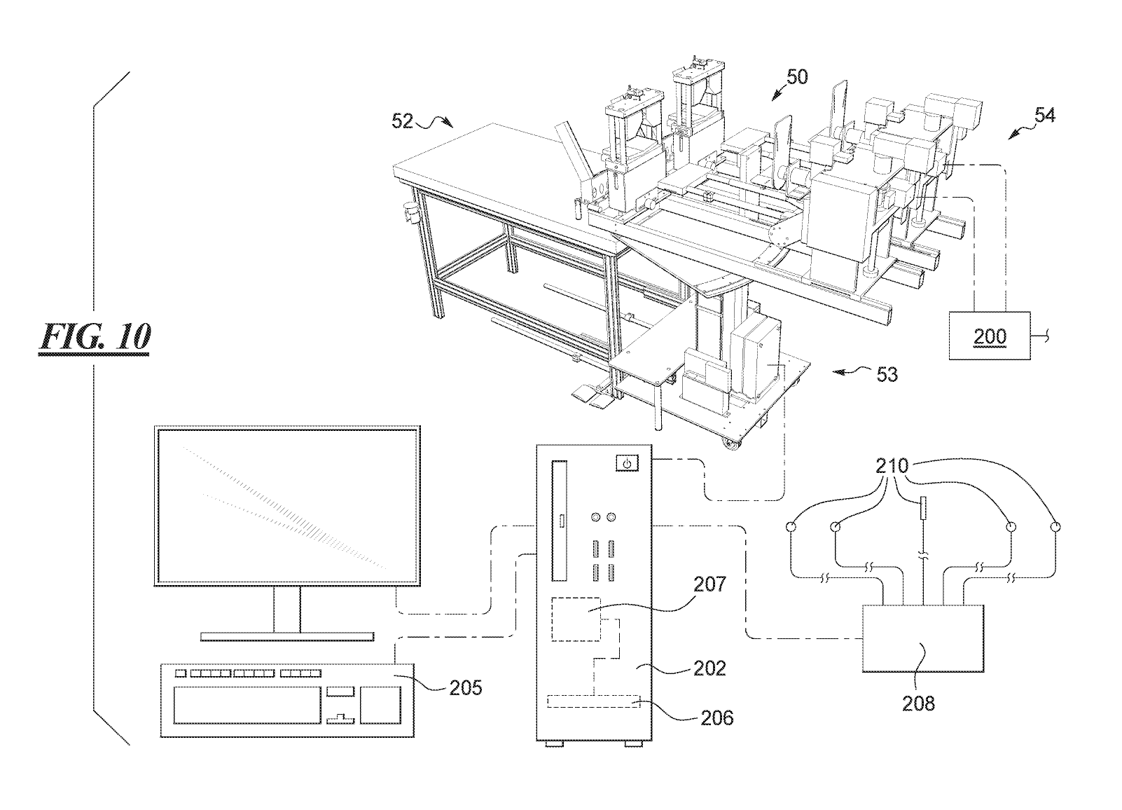

As shown in FIG. 10, the robot 54 of the RKT apparatus 50 can be part or a system and connected to a power source 200 to operate the robot. The power source 200 can be a typical 120/220 volt AC grid, a converted direct current power source, a stand-alone power source such as a generator or battery, or the like. The robot 54 of the RKT apparatus 50 can also be connected to a programmable electronic device or network of devices, such as a computer 202 or a computer network, a network server, or the like that are part of the system. In any case, the computer 202 can have or be connected with an input device 204, such as a keyboard, a user display 205, such as a monitor or screen, a memory 206, and a processor 207. The robot 54 and/or computer 202 can also be coupled to a sensor or tracking system 208. The tracking system 208 can utilize one or more individual sensors 210 that are configured to detect or determine spatial positioning or location of the sensor at a point in time. The types of sensors 210 and tracking system 208 can employ electromagnetic (EM) sensors, electromagnetic field (EMF) sensors, or other suitable sensor technology.

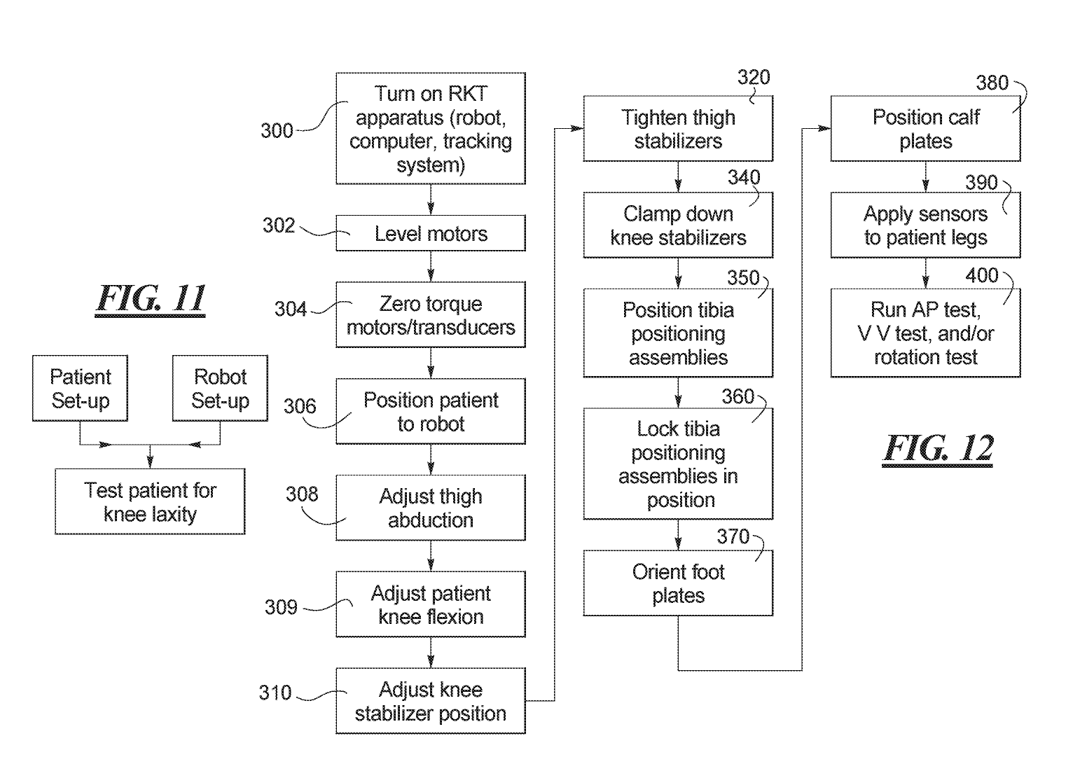

In the disclosed example, the X-, Y-, and Z-drives can be connected to and operable by the computer 202. The computer 202 can be programmed to receive and store load or torque data from the X-, Y-, and Z-drives 104, 106, 108 and to receive and store spatial position data from the sensors 210 and tracking system 208. The processor 206 can be programmed to calculate information and provide feedback related to knee laxity, based on the data. The information and feedback can be provided to the examiner on the display 205. The knee laxity information and feedback can relate to anterior-posterior movement, Varus-valgus movement, and/or tibia rotation movement, as described above. As represented in FIG. 11, the set-up of the patient relative to the RKT apparatus and particularly the robot 54 can be performed or specified as disclosed herein to aid in rendering the test data, information, and feedback more consistent and more accurate. Likewise, also as shown in FIG. 11, the set-up of the robot 54 prior to undertaking any testing can also be performed or specified to aid in rendering the test data, information, and feedback more consistent and accurate.

FIG. 12 shows a block diagram that is representative of a set-up method according to the teachings of the present disclosure. In this example, the method combines steps relating to setting up the patient relative to the RKT apparatus and setting up the robot 54 prior to testing. In other examples, the method may include only steps to set-up the patient relative to the RKT apparatus 50 and robot 54. Likewise, the method may include only steps to set up the robot 54 prior to testing.

With reference to FIG. 12, at block 300, the RKT apparatus 50 is turned on or powered up. In the disclosed example, to do so, the computer 202 including the applicable program, the tracking system 208 including the sensors 210, and the robot 54 are each started, turned on, or powered up. The objective of this step is to get the RKT apparatus up and running and to prepare the apparatus for use.

At block 302, the drives or motors of the robot 54 are leveled. In the disclosed example, to do so, the motors 110, 140, 160 of the corresponding X-, Y-, and Z-drives 104, 106, 108 can be precisely leveled relative to a horizontal or vertical reference or referencing a leveling device. In one example, a portion of the tracking system 208 can be used to precisely level the motors 110, 140, 160. Alternatively, the motors 110, 140, 160 can be leveled manually or mechanically such as by using an inclinometer. The objective of this step is to provide and define a consistent, repeatable starting point for the tibia positioning assembly 90 that can be achieved prior to each test using the RKT apparatus 50.

At block 304, the torque in each of the drives or motors is zeroed. In the disclosed example, to do so, each of the motors 110, 140, 160 of the drives 104, 106, 108 is zeroed. The motors 110, 140, 160 may thus be adjusted, positioned, or re-set to a condition where the torque transducers read zero torque or where the output shafts are under no torque. The objective of this step is to provide and define a consistent and repeatable starting condition, i.e., a neutral or zero torque starting point for each drive or motor prior to each test using the RKT apparatus 50.

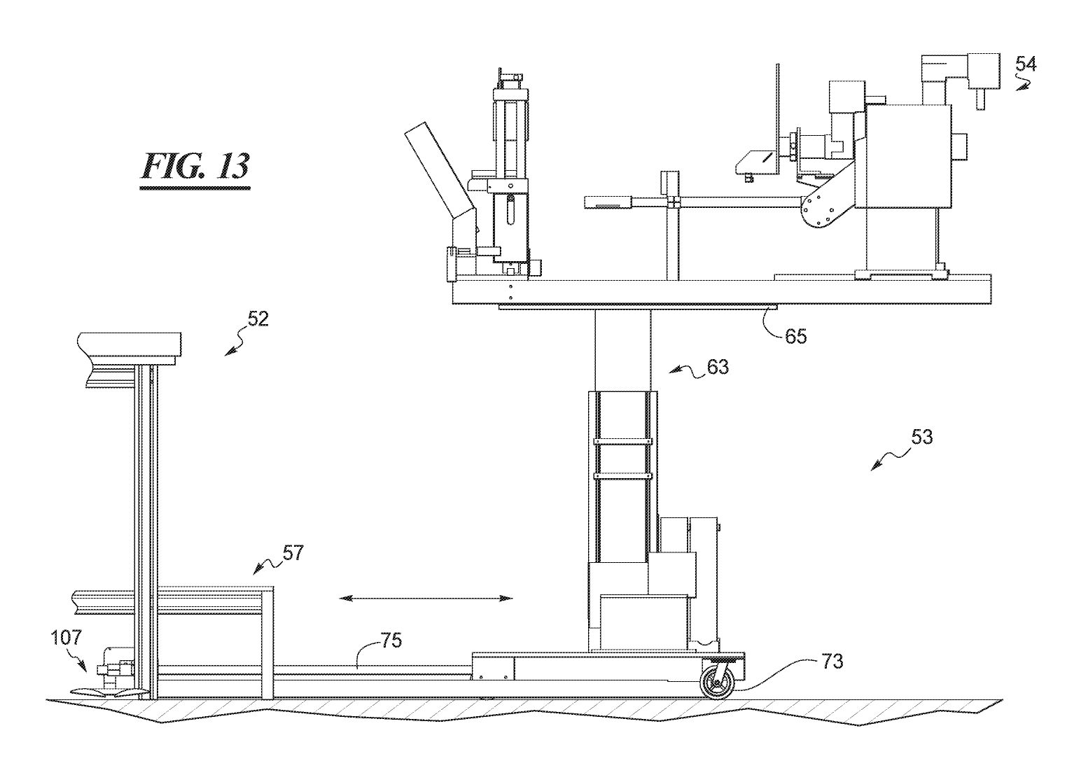

At block 306, the positioning system 53 is utilized to aid or assist a patient in getting up onto the table assembly 52 and in positioning the patient's lower extremities or lower legs relative to the robot 54 for testing and evaluation. In the disclosed example, as depicted in FIG. 13, the positioning system 53 and the robot 54 can be moved to an extended position slid away and spaced from the distal edge of the table assembly 52. The patient can then be situated in an orthostasis position between the robot 54 and table assembly 52.

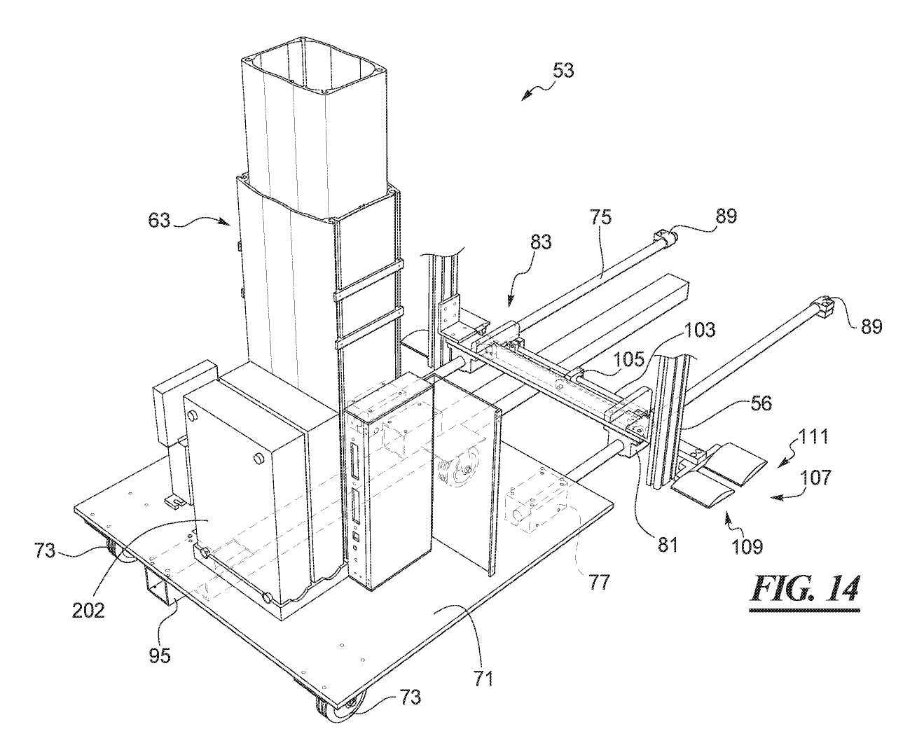

As depicted in FIG. 14, the positioning system 53 in the disclosed example includes a column base 71 that supports the column lift 63 in a vertical orientation. The column base 71 can also be used to support a programmable electronic device or network of devices, such as a computer 202 or a computer network, a network server, or the like for use with the robot 54 and to operate the column lift 63, the robot 54, and/or the RKT apparatus 50 and system components.

The column base 71 may be of any configuration that supports the column lift 63. In this example, the column base 71 has a flat panel 388 structure that may be supported on frame structure (not shown) that may provide rigidity and strength to panel of the column base. The column base 71 can include wheels 73 mounted to the underside of a portion of the column base 71, such as the flat panel 388. The positioning system 53 is thus supported on the wheels 73 for permitting the positioning system 53 to move in a horizontal direction, toward and away from the table assembly 52. The wheels 73 allow for the positioning system 63 to be moved away from the table assembly 52 to allow for patient ingress and egress. Additionally, the wheels 73 allow for horizontal movement of the robot 54, which is supported on the column lift 63, to adjust knee flexion when testing a patient. Other moveable supports in addition to the wheels 73 can be provided, such as a slide track or other structural elements. The column base 71 may further include one or more apertures 389 for directing cabling related to the robot 54 or computer 202 to a cable runner or channel 95 underneath the column base 71.

As described above, the column base 71 supports the column lift 63 of the positioning system 53. The column 63 lift includes an inner segment 390 and an outer segment 392 that are telescopically connected. The telescoping inner segment 390 functions to move vertically upward relative to the outer segment 392. The column lift 63 in this example carries a support plate 63 at the upper end of the inner segment 390 upon which the robot 54 rests or is mounted. The telescoping nature of the column lift 63 facilitates adjusting the robot 54 in a vertical direction, higher or lower, in relation to a height of the patient platform 58 of the table assembly 52. The vertical movement of the column lift 63 may be accomplished using a drive system (not shown) such as gear drive, screw drive, pneumatic or hydraulic system, or any other means for controlling vertical movement of the robot 54. The drive system may be controlled by a controller (not shown) to allow for measured vertical movements that can further be fixed and set at a specified height. These components, such as the controller and drive system may also be mounted to or supported on the column base 71 or the stationary outer segment 392 of the column lift 63.

As further depicted in FIG. 14, the column base 71 may be adjustable or movably connected to the table assembly 52 via a guide system or locking mechanism 83 to guide movement of the positioning system 53 as well as to fix the selected location and distance of the positioning system 53, including the column base 71, column lift 63, and robot 54 from the table assembly 52.

As depicted in FIGS. 14-16, the guide system or locking mechanism 83 has guide rods 75 that are fastened to the column base 71. The guide rods 75 can be elongate rods that have one end rigidly fastened to the underside of the column base 71 via brackets, mounts, or couplers 77. The other ends or distal free ends of the guide rods 75 are slidably connected to the table assembly 52 via bearings or slide elements 81. The free ends of the guide rods 75 extend through and past the slide elements 81. The guide rods 75 thus extend beyond the column base 71 and underneath the table assembly 52. In another example, the guide rods 75 may be fixed to the table assembly 52 and slidably connected to the column base 63, if desired. The guide rods 75 are of sufficient length such that the positioning system 53 may be moved away from the table assembly 52 far enough to allow a patient to stand between the table assembly 52 and the robot 54 when the positioning system 63 is pulled away from the table assembly 52 as shown in FIG. 13. An optional stop 89 is affixed to the free ends of the guide rods 75 to limit the distance that the robot 54 can move away from the patient assembly in the horizontal direction.

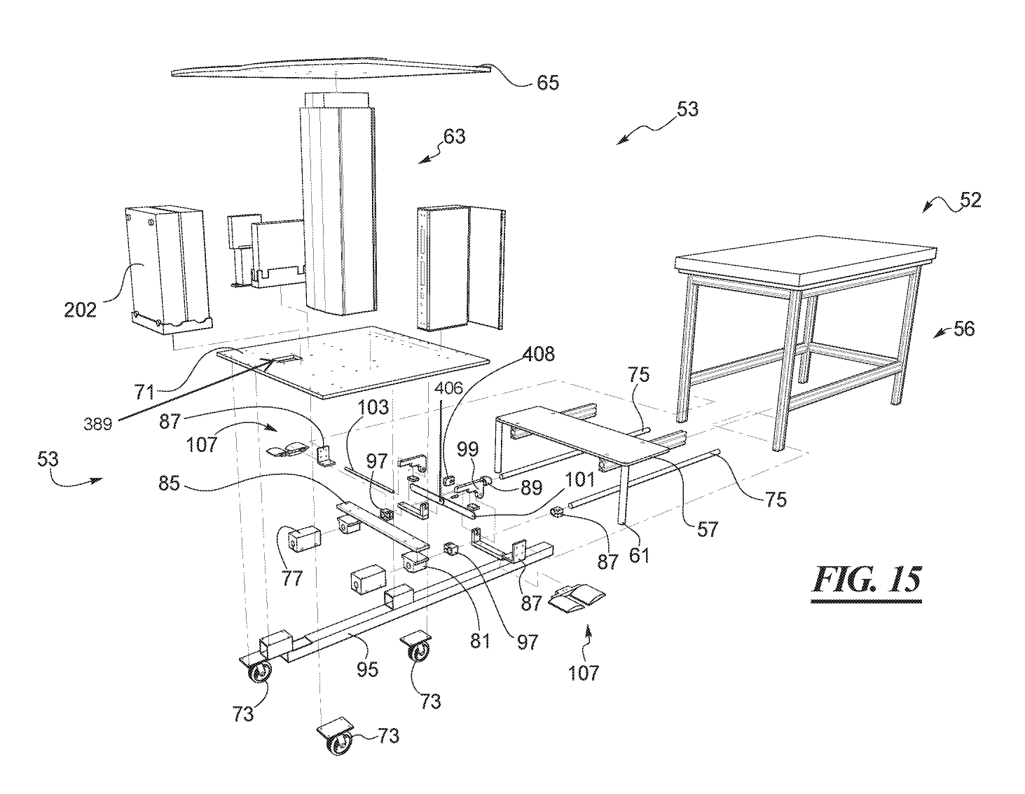

With reference to FIGS. 15-17, the guide system or locking mechanism 83 includes pinch blocks 97 that interact with the guide rods 75 to lock the positioning system 53 in place in a selected horizontal position, particularly for testing and evaluation of a patient. In this example, a mounting bar 85 is or can be mounted to the base 56 of the table assembly 52, such as between legs of the base using a pair of L-shaped brackets 87. The aforementioned bearings or slide elements 81 are attached to an underside of the mounting bar 85. Pivot brackets 99 have one leg attached to a top side of the mounting bar 85. An opposite end of each pivot bracket 99 defines a pivot point P spaced from and below the mounting bar 85.

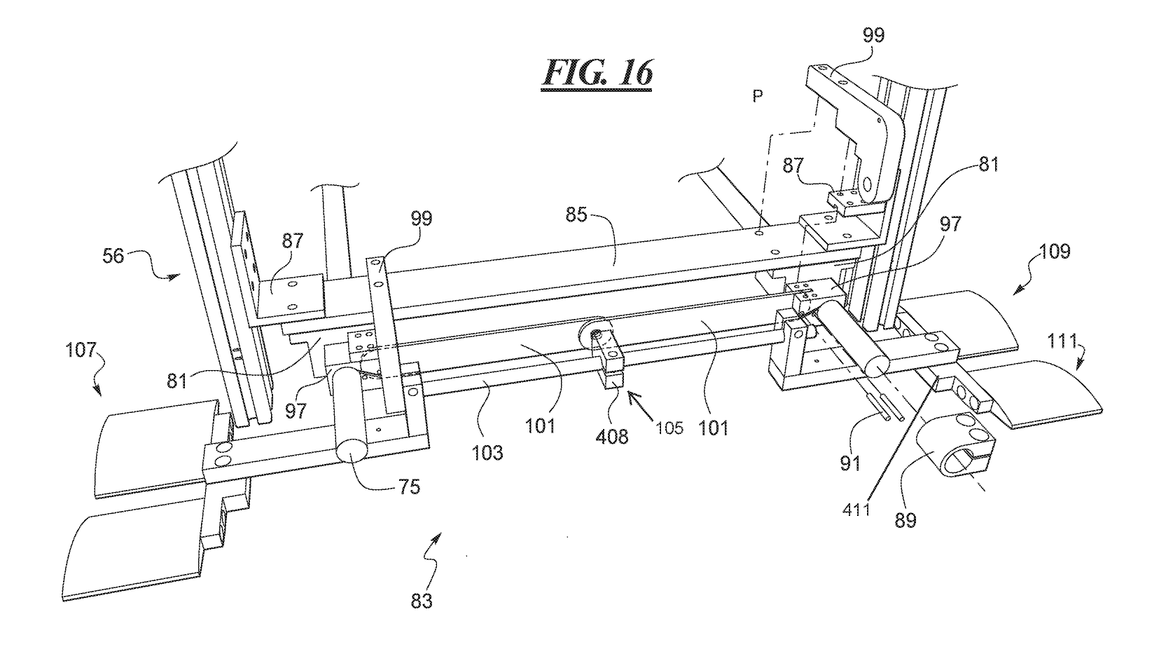

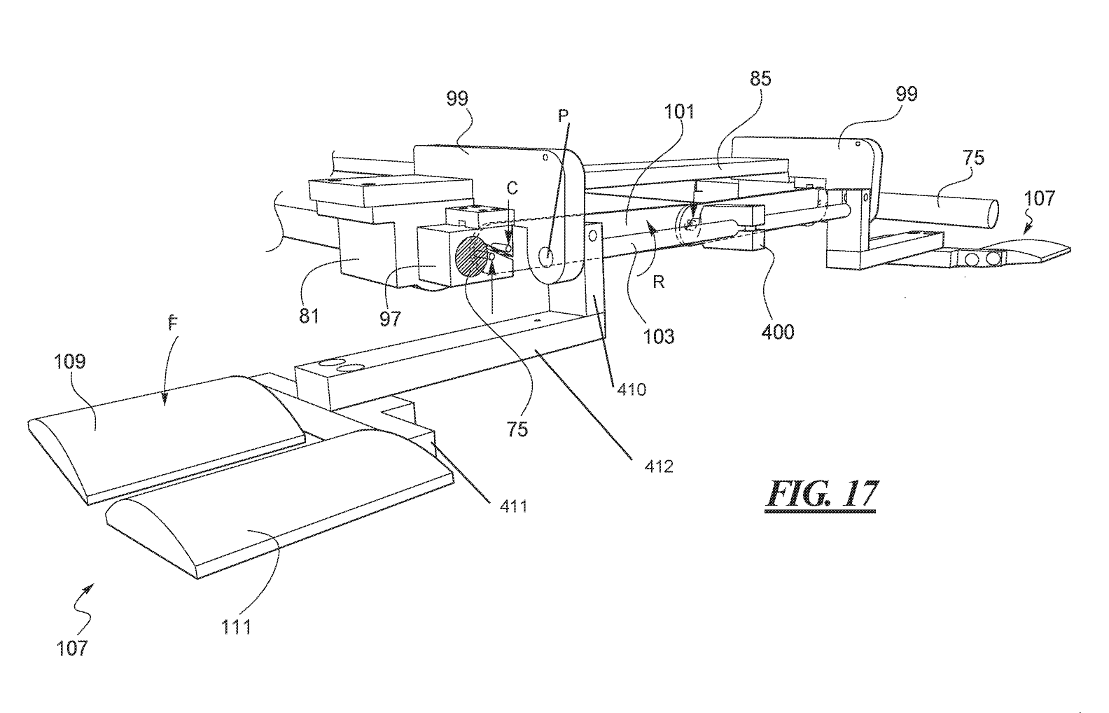

As depicted in FIG. 16, the guide system or locking mechanism 83 includes two of the pinch blocks 97, one located on each side of the mechanism 83 and table assembly 52. This is so that, as described below, the clinician can operate the locking mechanism 83 from either side of the RKT apparatus. It is contemplated, however, that the locking mechanism 83 may function with only a single pinch block, such that the locking mechanism is operable only from one side of the RKT apparatus. The pinch blocks 97 can be generally cube shaped and can include a through aperture 402. The aperture 402 in each of the pinch blocks 97 is sized to accept one of the guide rods 75 therethrough. The pinch blocks 97 can further include an angled slit 400 extending through the bodies of the pinch blocks 97 from the aperture 402 to the outer surface of the pinch blocks 97. The slits 400 enable the apertures 402 of the pinch blocks 97 to expand or contract, as further discussed below. Each pinch block 97 can also include two through bores or holes 404 that extend parallel to the apertures 402. One of the holes 404 is positioned on each side of the slit 400.

The locking mechanism 83 also includes a pair of locking bars 101 that each are an elongate blade-like element. The locking bars 101 each have first or outer ends that are attached to a respective one of the pinch blocks 97. Each outer end includes a pair of holes that align with the openings 404 in the pinch blocks 97. Dowel rods or pins 91 are received through the holes in the locking bars 101 and the openings 404 to secure the bars to the pinch blocks 97. It should be noted that the holes in each of the locking bars 101 and the pins or dowel rods 91 received therein are positioned one on each side of the slit 400 because the openings 404 in the pinch blocks 97 are so positioned. The second or inner ends of the locking bars 101 each include a key hole to commonly connect the locking bars 101 to a locking key 105. The openings in the locking bars 101 may be slightly oval in shape to fit a like shaped pin or stud 406 to secure the locking key 105 to the locking bars 101. The attachment of the locking bars 101 to the pinch blocks 97 as depicted in FIG. 17 and the existence of the slits 400 are configured to cause, by slight rotation in one direction of the locking bars, to reduce the size of the apertures 402 through the pinch blocks 97 to clamp around and onto the guide rods 75. In the alternative, by slight rotation in the other direction of the locking bars 101, the size of the apertures 402 through the pinch blocks 97 can be expanded by acting on the pinch blocks 97 to release pressure on the guide rods 75.

The locking key 105 can include the stud 406 and a coupler portion 408. The stud 406 of the locking key 105 extends through the openings on the inner or second ends of the locking bars 101. The locking key 105 facilitates the clamping or releasing motion of the locking bars 101 imparted to the pinch blocks 97 as described above and further below. An elongate pivot rod 103 extends generally from one side of the locking mechanism 83, through the coupler portion of the locking key 105, to the other side of the locking mechanism 83. Each end of the pivot rod 103 extends through and is connected to a C-clamp 410, each of which is connected to a separate pedal assembly 107 positioned adjacent opposite portions of the frame 56 on the table assembly 52. Exposed end segments of the pivot rod 103 are received in openings that form the pivots P of the respective pivot brackets 99. The coupler portion 408 of the locking key 105 can be a C-clamp element that can clamp tightly onto the pivot rod 103. Rotation of the pivot rod 103 about its lengthwise axis can thus cause the locking key 105 to rotate. This in turn will raise or lower the stud 406 of the locking key 105, which in turn will raise or lower the second ends of the locking bars 101, as depicted in FIG. 17.

As depicted in FIGS. 16 and 17, the locking mechanism 83 includes two of the aforementioned pedal assemblies 107 that are manipulated to lock and unlock the locking mechanism 83. The pedal assemblies 107 each include a locking pedal 109 and an unlocking pedal 111. The locking pedal 109 and unlocking pedal 111 are each fixed to and carried on a rigid link 411. The rigid link 411 is oriented transversely and connected to an arm element 412 at one end of the arm element. The other end of each arm element 412 is connected to a respective one of the C-clamps 410 of the pedal assemblies 107. Each of the C-clamps 410 is also fixed to its respective end of the pivot rod 103. The C-clamps 410 are pivoted about the lengthwise axis of the pivot rod 103 by pushing down on one of the pedal assemblies 107, which in turn rotates the pivot rod 103 to raise or lower the locking key 105, causing the second ends of the locking bars 101 to be raised or lowered.

In using the locking mechanism 83 to fix the location of the positioning system 53, a clinician may step down on the locking pedal 109 of either of the pedal assemblies 107 in the direction of the arrow F. Doing so will twist the arm elements 412 and rotate the C-clamps 410 causing the pivot rod 103 to rotate in the direction of the arrow P. Rotating the pivot rod 103 rotates the locking key 105 to drive the stud 406 and locking bars 101 downward in the direction of the arrow L in FIG. 17). The downward movement of the locking key 105 and the second ends of the locking bars 101 rotates the first ends of the locking bars 101. This in turn changes the position of the two dowel rods or pins 91 relative to one another on each of the pinch blocks 97. Rotation of the locking bars 101 in the R direction draws the dowel rods 91 toward each other in the direction of the arrows C in FIG. 17, thereby closing the gap of the slits 400 and reducing the diameter of the apertures 402 in the pinch blocks 97. Reducing the diameter of the apertures 402 causes the pinch blocks 97 to squeeze the guide rods 75, thereby fixing the guide rods and preventing horizontal movement of the column base 71, the positioning system 63 and robot 54. Alternatively, a clinician can step on the unlocking pedal 111 of either of the pedal assemblies 107. Doing so will twist the arm elements 412 in the opposite direction and rotate the C-clamps 410 causing the pivot rod 103 to rotate in the direction opposite that of the arrow P. Rotating the pivot rod 103 in the other direction rotates the locking key 105 and moves the stud 406 upward. The upward movement of the locking key 105 raises the second ends of the locking bars 101, which in turn pivots the first ends of the locking bars in the opposite direction. The first ends of the locking bars 101 will act on the pinch blocks 97 to expand the slits 400 and increase the diameter of the apertures 402, releasing the guide rods 75 to slide. This in turn allows for horizontal movement of the positioning system 63 and robot 54 (FIG. 16).

As shown in FIG. 14, there are two guide rods 75 and one locking mechanism 83 operable from either side of the RKT apparatus by virtue of having two of the pedal assemblies 107. However, the RKT apparatus may function with only one guide rod and only one of the pedal assemblies. Further, the locking mechanism and guide rods can include additional features, though not disclosed or described herein, that may be used to assist in moving the robot and fixing the position of the robot relative to the table or to otherwise enhance patient comfort or improve performance of the RKT apparatus. Alternatively, the locking mechanism and/or guide rods may vary in shape, configuration, operation, and construction, as desired to move the robot and/or fix the position of the robot relative to the patient support. Other moveable supports in addition to or as a replacement for the guide rods 75 can include a track configuration below the column base 71 for rolling members (i.e. ball bearings) that would travel in or on the track. The horizontal movement of the column base 71, column lift 63, and robot 54 can also be controlled by a drive system as described above relating to the vertical movement of the column lift 63.

In positioning the patient on the table assembly 52, the patient may use the step 57 to climb onto the padded surface 60 of the platform 58 on the table assembly 52. The patient may next sit on the distal edge of the table assembly 52 with their legs hanging over the edge. The patient may then lay back in a supine position with their trunk supported by the patient platform 58 in a supine position and pull their knees towards their chest. The robot 54 may then be slid or rolled toward the table assembly 52 using the wheels 73 and guided by the released guide rods 75. When the robot 54 is in the desired position relative to the patient, either locking pedal 109 of the locking mechanism 83 may be depressed to lock the horizontal position of the robot 54. The column lift may also be adjusted up or down to a preferred height for further positioning the patient relative to the robot 54. The vertical movement of the column lift 63 may also be fixed or locked to retain the desired vertical position of the robot 54.

The patient may then extend their legs over the robot 54. The patient is then positioned with their legs adjacent the tibia positioning assemblies 90. First, the knee stabilizers 74 are manipulated to remove the upper clamping element 78 so as to permit the legs of the patient to drop down onto the lower knee clamping element. The legs of the patient are then positioned so that the joint line of each knee is slightly in front of the front plane, i.e., the foot facing side of the corresponding knee stabilizer 74, so as to provide clearance between the tibia and the clamping element 78. The objective for this step is to provide a consistent, repeatable target position in the Z-axis direction for the knees of a patient with respect to the thigh and knee stabilizers 70, 74. In this position, the lower legs of a patient are also free to bend at the knee forward of the lower knee clamping element 78 while the lower femur of each leg is fully supported on the pad 79 of the lower knee clamping element.

At block 308, the abduction angle of the patient's femurs is adjusted relative to their hips. In other words, the patient moves or is positioned on the table assembly 52 and on or in the tibia positioning assemblies 90 so that their femurs are at a desired abduction angle. In one example, the tibia positioning assemblies 90 may be pivotable or movable in order to adjust or change the angle between the two assemblies relative to a mid-line of the apparatus and/or the patient. This adjustment can be done in order to adjust the abduction angle of the patient's femurs so that their femurs are neutrally aligned with their hips. Alternatively, and in this example, the tibia positioning assemblies 90 may be in a fixed abduction orientation, such as at a fixed 30-degree angle relative to one another, as noted above. The thigh stabilizers 70 may then be adjustable sideways as mentioned further below so that the patient's femurs can be neutrally aligned with their hips. The objective of this step is to position the patient's femurs in a consistent, repeatable, and comfortable manner relative to the robot 54. The desired position is to have the femurs neutrally lined up with the patient's hips so as to limit stress on the patient's upper legs and hips during a test and to create a repeatable and consistent orientation of the lower legs relative to the femurs of the patient.

At block 309, the position of the robot 54 is adjusted relative to the patient's trunk and table assembly 52 in the horizontal and vertical direction using the positioning system 53 to position the patient's knees in a desired degree of flexion. Here, the vertical movement of the column lift 63 and horizontal movement of the locking mechanism 83 may be done simultaneously or independently to adjust the degree of flexion in the patient's knee. Using the column lift 63 of the positioning system 53, the robot 54 can be adjusted up or down to raise or lower the knees of the patient. The robot 54 may be moved by also rolling or sliding the positioning system 53 towards or away from the table assembly 52 to retract or extend the legs of the patient. Utilizing the column lift 63 to raise or lower the robot 54 and the locking mechanism 83 of the positioning system 53 can allow the clinician to position the patient's knees in the desired flexion in a range of 0 to 90 degrees. Once the desired knee flexion is reached, the positioning system 53 can fix the position of the robot by locking the locking mechanism 83 and braking, locking, or retaining the column lift 63 at the desired height.

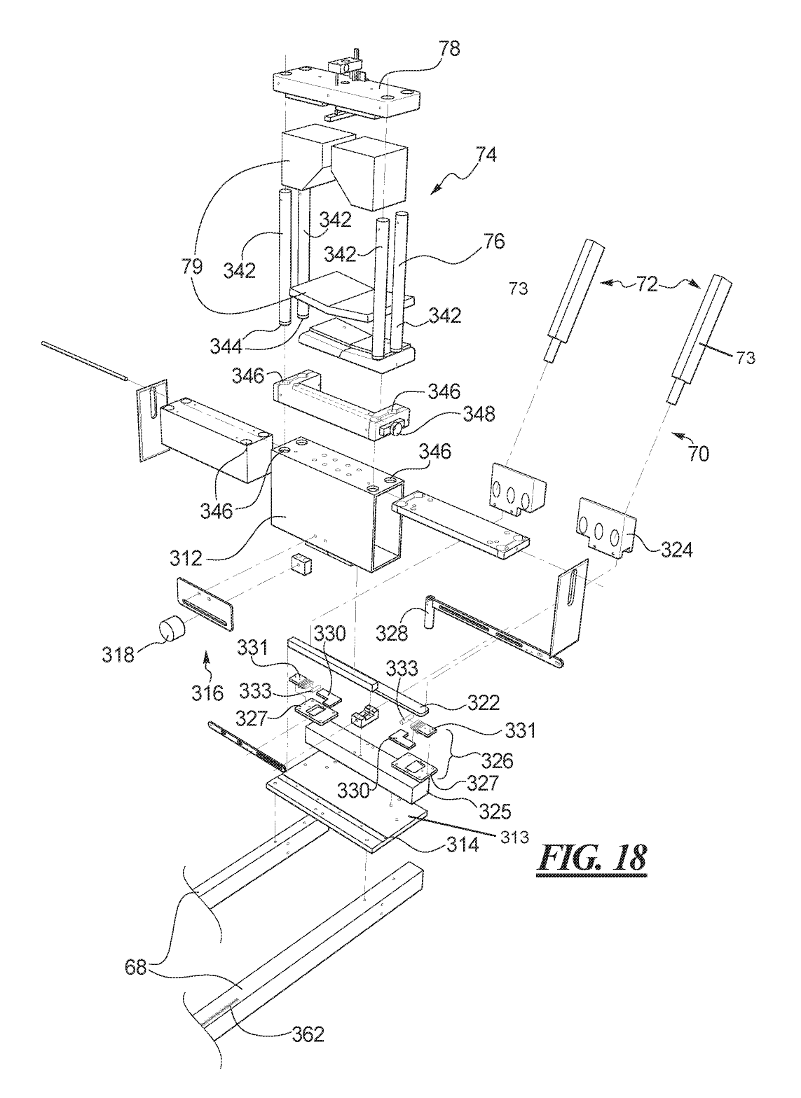

At block 310, the patient's knees are centered relative to the respective knee stabilizers 74. In the disclosed example, as shown in FIG. 18, each knee stabilizer 74 is mounted on or to a support base 312, which is positioned under and coupled to the lower knee clamping element 78. The support base 312 is mounted on an adjustment or mounting plate 313 that includes or defines a slide track 314 and that is carried by part of the RKT apparatus 50. In this example, the mounting plate 313 forms a cross-member traversing the rails 82 on the sub-frame 68. However, the mounting plate 313 can instead be a separate component mounted to the sub-frame 68, a cross-member 84, or another part of the RKT apparatus 50. The support base 312, and thus the knee stabilizer 74, is side-to-side adjustable along the slide track 314. The support base 312 and/or slide track 314 can incorporate a locking element 316 that is configured to selectively secure or release the knee stabilizer 74 relative to the slide track.

In the disclosed example, to center the knee stabilizers 74 on the patient's knees, one can release the locking elements 316 and slide the knee stabilizers side-to-side along the respective slide track 314. The knee stabilizers 74 can be moved side-to-side to center the corresponding posterior knee pads 79 on the lower knee clamping elements 78 under the knees of the patient. Though not specifically described herein, the locking elements 316 can include a knob 318 that is manipulated to lock or release the knee stabilizers 74 relative to the slide track 314. The construction of the support base 312, mounting plate 313, slide track 314, and locking elements 316 can vary considerably and still function as intended to provide side-to-side adjustability of the knee stabilizers 74. One objective of this step is to define a consistent and repeatable position for the patient's knees relative to the tibia positioning assemblies 90 generally in the X-axis direction. Another objective of this step is to center the patient's knees within the knee stabilizers 74 so that, when ultimately clamped onto the knees of the patient, each knee is centered among the pads 79 and thus securely retained in position to prevent movement of the femur and patella once clamped in the respective stabilizer.

At block 320, the thigh immobilizers 70 are adjusted to secure the patient's femurs in place. In the disclosed example, as shown in FIG. 18, each thigh immobilizer 70 has a primary mechanical adjustment device. Each thigh immobilizer 70 is mounted to the guide rails 82 via a support block 325 mounted to the top portion of the mounting plate 313. Mounted on top of the support block 325 is a locking mechanism 326 used to adjust and fix the respective thigh immobilizer 70 in place independently. Each thigh immobilizer 70 has two of the thigh clamping elements 72. Each clamping element 72 has a truck 324 that carries a paddle 73 extending upward from the truck.

In the disclosed example, the clinician can release the locking mechanisms 326 and slide the thigh clamping elements 72 and trucks 324 along a respective locking bar 322 by pulling or pushing on handles 328 to release the locking mechanisms. Though not specifically described herein, the construction of the locking mechanisms 326 can vary considerably and still function as intended to provide side-to-side independent adjustability of the thigh clamping elements 72 on each of the thigh immobilizers 70.

As shown in FIG. 18, and as described above, the trucks 324 and thigh clamping elements 72 can optionally include a secondary distinct mechanical adjustment device as well. This feature can aid in allowing the thigh immobilizers 70 to accommodate a wider range of patient leg sizes from small children to large adults. In this example, each truck 324 has three bores 337 that are spaced apart along the width of the truck 324 and open to the top surface of the truck. Each paddle 73 has a corresponding peg or pin 334 protruding downward from the body of the paddle. The peg 334 of each paddle can be selectively inserted into any one of the three bores 337 in the corresponding truck 324. By choosing one of the three bores, and without moving the trucks 324, the adjacent paddles on one of the thigh immobilizers 70 can be mounted to the trucks 324 in nine different positional arrangements. Using the outer most bores 337, the paddles can be mounted further apart from one another. Using the inner most bores 337, the paddles can be mounted closer together. Using a combination of one inner bore 337 and one outer bore, or either or both of the center bores, the paddles can be mounted in an intermediate spacing. Depending on which inner and which outer bores or which center bore is selected, the paddles can be shifted to the left or to the right, if desired or needed, also without having to move the trucks 324. This secondary adjustment scheme allows for greater versatility in setting up a patient. Any type of locking mechanisms, such as a cam lock type device, can be used to also secure the pegs 334 in the bores 337, if desired, or a separate retention means, if any, may also be used to retain the paddles to the trucks 324.

Once the patient's knees are correctly positioned, according to the step at block 306, and the knee stabilizers 74 are centered according to the step at block 310, the thigh immobilizers 70 can be adjusted, set, and clamped onto the patient's thighs. With handles 328 of the locking mechanisms 326, the clamping elements 72 of each thigh immobilizer 70 can be independently adjusted so that the respective thigh clamping elements forcibly contact the sides of the patient's thigh. The handles 328 on the locking mechanisms 326 are then released, securing the thigh immobilizers 70 in place. Each thigh clamping element 72 should be positioned or secured such that the medial and lateral clamping elements apply substantially equal pressure to the thigh. One objective of this thigh clamping step is to permit a consistent and repeatable position for the patient's thighs relative to the tibia positioning assemblies 90, also generally in the X-axis direction. Another objective of this thigh clamping step is to then securely clamp the patient's thighs in place with the thigh immobilizers 70. During testing, it is desirable that the femur position for each leg of a patient is securely retained to prevent sideways movement and femoral rotation once the thigh immobilizers 70 are adjusted and locked in place.

At block 340, each knee stabilizer 74 is clamped onto the patient's knee or patella. In the disclosed example, as depicted in FIG. 4, the framework 76 of each knee stabilizer 74 can include a pair of guide posts 342 on each side of the stabilizer. The guide posts 342 can be fixed to the upper knee clamping element 78 and can depend down from the element. Free ends 344 of the guide posts can be received in and slide through a corresponding pair of holes 346 on each side of the lower knee clamping element 78. The upper and lower clamping elements 78 are adjustable vertically relative to each other, as noted above, by sliding the upper clamping element 78 and guide posts up and down relative to the lower clamping element 78, which is fixed to the support base 312. A fixing screw 348 in this example extends transversely into each side of the lower clamping element 78 between the pair of holes. The fixing screw, when rotated in one direction can reduce the diameter of the holes to clamp onto and lock guide posts 342 and, when rotated in the opposite direction, can increase the diameter of the holes to release the guide posts. With the guide posts 342 released, the upper knee clamping elements 78 (and guide posts) can be removed from the lower knee clamping element 78 so that the patient's knees can be readily positioned on the lower clamping elements, as noted for the step at block 306. Once the knees are properly positioned after the step at block 306, the upper knee clamping element 78 can be replaced on the lower knee clamping element 78 any time before block 340.

At this point, the locking elements 316 on the knee stabilizers 74 are still released so that the knee stabilizers 74 are free to slide or move along the slide track 314. Also at this point, the upper knee clamping element 78 should now be or should already have been reinstalled on the lower knee clamping element 78. The upper knee clamping element 78 is then clamped downward so that the pads 79 on the upper knee clamping element press down against the patella of the knee. The downward clamping force should achieve a predetermined or desired force, such as 30 lbs., and equal pressure should be applied to both the medial and lateral sides of each knee stabilizer 74. The knee stabilizers can then be secured in this clamping condition. In this example, the fixing screws can be rotated to secure the guide posts 342. A force gage or other suitable method and/or device can be used to achieve the desired downward clamping force applied by the knee stabilizers on each patella of the patient. Once the knee clamping elements 78 are clamped and locked, the knee stabilizers can then be locked in place on the slide track 314 by actuating the knobs 318. The objective of this knee clamping step is to securely clamp the patient's knee at the patella in with knee stabilizers 70. During testing, it is desirable that the lower end of the femur and the patella are securely retained to prevent vertical movement at the patella once the knee stabilizers 74 are adjusted, clamped down, and locked.

At block 350, the patient's feet are placed against the contact surfaces 94 and heel stops 93 of the foot plates 92. In the disclosed example, the tibia positioning assemblies are drawn toward the patient's feet by sliding the assembly along the tracks 80 on the sub-frames 68. In an alternative example, the drive system may be stationary and only the foot plates 92 may be adjustable along the Z-axis to contact the patient's feet. Once the feet are in contact with the two plates 92, the tibia positioning assemblies 90 are in a testing position relative to the patient's feet and lower legs. When the feet are properly positioned, appropriate straps (not shown) can be used to secure the feet to the foot plates. One objective of this step is to provide a consistent and repeatable mechanism to properly position the tibia positioning assemblies 90 along the sub-frames 68 relative to a specific patient. Another objective of this step is to secure the patient's feet to the foot plates and thus to the drive system of the tibia positioning assemblies.

At block 360, the tibia positioning assemblies 90 are locked in place. In the disclosed example, each tibia positioning assembly 90 can be locked in the position achieved at the step of block 350. For example, though not depicted herein, a lock pin for each tibia positioning assembly 90 or on the sub-frame 68 can be inserted into a groove or hole on the other. This will lock the tibia positioning assemblies 90 at the adjusted position accommodating the particular patient being set up. A ruler 362 or other indicia or markings may be provided on or along one of the lengthwise parts of each sub-frame 68, such as along one of the rails 82 (see FIG. 5). The rulers 362 can be configured to identify the length of the lower legs of the patient being set up, based on the position of the tibia positioning assemblies 90 along the tracks 80 or the sub-frames 68. This measurement can be recorded for each specific patient and can then be utilized to set up the robot 54 for a particular patient each time the patient is tested. This helps to ensure that the RKT apparatus is set up the same way for the same patient. The objective of this step is to aid in providing a fixed, consistent, and repeatable set-up position for the tibia positioning assemblies for each patient.

At block 370, the patient's feet are rotated to a desired initial rotational orientation. In the disclosed example, each foot plate 92 can be manually rotated to a desired position determined by the orientation of a part of the patient's foot or a part of the foot plate. For example, the patient's foot could be positioned with the toes up and perpendicular to the floor beneath the RKT apparatus. More specifically, the starting orientation may be to orient the second toe on each foot point vertically perpendicular to the floor. This initial foot rotation position can instead be established by moving the Z-axis motor 160 into a neutral zero-torque position to find a true resting position for the patient's feet. The objective of this step is to define a consistent and repeatable starting orientation for the foot plates 92.

At block 380, each tibia rod device 96 is properly positioned under the patient's calves. In the disclosed example, each tibia rod device 96 can be length adjustable to retract or extend the calf plate 100 to a desired position under the corresponding calf of the patient. Once in the desired position, the calf plate is in a testing location or an AP test location relative to the patient's leg. A ruler or other indicia or markings (not shown) may be provided along part of the tibia rod device 96 to help determine the proper or desired position for the calf plate 100 (see FIG. 6). For example, the slider segment 98b of one of the tibia rods 98 can include the ruler 362 or markings that correlate with the ruler 362 on the tibia positioning assemblies 90. If the desired positon of the calf plate 100 for each patient is to be three-quarters (¾) of the way up the leg from the patient's heel, the ruler (not shown) can be a ¾ scale version of the ruler 362, which defines the patients leg length. Thus, by selecting the same measurement on both rulers 362, the position of the calf plate 100 is assured on each tibia positioning assembly 90 for each patient. Such measurements help to ensure that the patient set-up is as consistent as possible. The objective of this step is to provide a mechanism to ensure repeatable and consistent positioning of the tibia rod device 96 so that the AP test is always conducted at the same relative location on each patient's legs.

At block 390, tibial sensors 210 are placed on the patient's legs. In the disclosed example, sensors 210 are positioned on the flat region of the bone that is just medial to the tibia tubercle on each leg. The sensors 210 are then strapped into place at this location. The location is selected for the sensors 210 because this region has the least amount of soft tissue between the sensor and the bone. This location will thus help during testing to limiting the degree of movement of the sensors caused by the soft tissue moving relative to bone. In one example, round sensor holders can be used to retain each sensor 210 in order to inhibit or prevent the sensors from rocking, due to compression of the calf muscle during testing.

Though not mentioned above, a ruler or other indicia or markings can be provided on other parts of the RKT apparatus to indicate specific positions of particular parts of the robot 54 after setting up a specific patient and the robot for testing. Rulers can also be provided on a portion of the thigh stabilizers 70, the knee stabilizers, and/or guide posts 342. In another example, a ruler can be provided on a portion of the tibia positioning assemblies 90, such as on the pivot plate 150, to indicate Varus-Valgus starting position. In yet another example, a marking scale may be provided on a portion of the Z-axis drive to indicate the position of the foot plates 92. Any such markings, indicia, or rulers can be used to record specific set-up parameters for a given patient that are repeatable from test to test each time the patient is set up for testing.

Additional set-up procedures may be utilized during testing or prior to testing in addition to those discussed above. For example, during AP testing, one or more straps may be utilized to secure the patients legs to the tibia rod devices 96. This may be to ensure that the tibia rod devices can both push up in an anterior direction on the patient's legs and pull down in a posterior direction on the patient's legs during testing. Once the AP test is completed, these straps may be removed and the tibia positioning rods can be moved out of the way prior to conducting a rotation test or a Varus-valgus test on the patient. In another example, during a Varus-valgus test, additional pads can be pushed into the knee stabilizers between the medial and lateral sides of the patient's knees and the framework 76. Such pads may help to minimize medial or lateral movement of the knee under the clamp and minimize axial rotation during the Varus-valgus test.

The patient and methods disclosed herein may vary from the examples shown and described. One or more of the specific steps may be performed as described but in a different order. Specific steps may be eliminated or altered and additional steps may be added. The design of the RKT apparatus may vary considerably from the example disclosed herein. As the design of the robot or apparatus varies, so may the steps vary, the order of the steps change, the number of steps change, and/or the specific details of the steps be altered or modified. The specific designs of the knee and thigh stabilizers may change, whether related to how the stabilizers are assembled, constructed, adjusted, locked, released, or the like. Likewise, the specific designs of the axis drives and/or the overall tibia positioning assemblies may also change.

The disclosed set-up procedures have been developed and are being refined in order to aid in reducing error and inconsistency in the test results and the underlying procedures. Some of the disclosed set-up steps are for setting up the patient position relative to the robot. Some of the disclosed set-up steps are for setting up the robot itself. However, all of the steps are conceived to aid in rendering the test procedures and results more accurate and more consistent. According to the disclosure, any patient can be set up relative to the robot in substantially the same way as any other patient. This can make knee laxity data acquired for different patients more directly comparable. According to the disclosure, a given patient can be set up relative to the robot in substantially the same way each time the patient is tested. This can make that patient's test results more relevant when comparing one test to the next. According to the disclosure, the robot can be set up using substantially the same procedure for any patient, other than where patient specific settings are known. This can reduce the amount of error that might otherwise be introduced into any given test.

Many modifications to and other embodiments of the disclosed RKT apparatus, components, methods, uses, and the like set forth herein may come to mind to one skilled in the art to which the invention pertains upon reading this disclosure. Therefore, it is to be understood that the inventions are not to be limited to the specific embodiments and combinations disclosed and that modifications and other embodiments and combinations are intended to be included within the scope of the appended claims. Although specific terms are employed herein, they are used in a generic and descriptive sense only and not for purposes of limitation.

Specific combinations of features, components, aspects, procedures, methods, steps, processes, and arrangements of and for the disclosed RKT apparatus and set-up are disclosed herein. However, one having ordinary skill in the art will understand that each feature, component, aspect, procedure, method, step, process, and arrangement may be used independently or in other combinations not specifically disclosed.

Although certain RKT apparatuses and methods have been described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A robotic knee testing apparatus comprising:
a robot configured to support a leg of a patient;
a patient support configured to support the patient thereon; and
a positioning system adjustably connected to the patient support and movable relative to the patient support,
wherein the positioning system is movable to adjust a position of the robot so as to allow the patient to be situated in an orthostasis position between a distal edge of the patient support and the robot.

2. A robotic knee testing apparatus of claim 1, wherein the positioning system is movable to adjust a vertical position of the robot.

3. A robotic knee testing apparatus of claim 1, wherein the positioning system further comprises a column lift supporting the robot, the column lift being operable to adjust a vertical position of the robot relative to the patient support.

4. A robotic knee testing apparatus of claim 3, wherein the column lift includes an inner segment and an outer segment telescopically connected relative to one another.

5. A robotic knee testing apparatus of claim 1, wherein the positioning system is movable to adjust a horizontal position of the robot toward and away from the patient support.

6. A robotic knee testing apparatus of claim 1, wherein the positioning system further comprises a guide such that a portion of the positioning system is slidable relative to the patient support.

7. A robotic knee testing apparatus of claim 6, wherein the guide includes at least one guide rod with a first end fixed to a base of the positioning system and a second end.

8. A robotic knee testing apparatus of claim 7, wherein the second end of at least one guide rod is slidably coupled to a portion of the patient support.

9. A robotic knee testing apparatus of claim 6, wherein the guide includes a pair of the guide rods.

10. A robotic knee testing apparatus of claim 1, wherein the positioning system further comprises a locking mechanism to lock the positioning system in a selected position so as to fix the position of the robot relative to the patient support.

11. A robotic knee testing apparatus of claim 1, further comprising a step tread attached to the patient support and located between the patient support and the positioning system.

12. A robotic knee testing apparatus of claim 1, wherein a portion of the robot is positioned beneath a knee of the leg when the patient is situated in a supine position on the patient support.

13. A robotic knee testing apparatus comprising:
a robot configured to evaluate a knee of a patient;
a patient support configured to support the patient thereon; and
a positioning system adjustably connected to the patient support and movable relative to the patient support,
wherein the positioning system is movable to adjust a vertical position of the robot relative to the patient support.

14. A robotic knee testing apparatus of claim 13, wherein the positioning system is movable to reposition the robot spaced from a distal edge of the patient support to allow the patient to pass between the distal edge of the patient support and the robot to get up onto the patient support.

15. A robotic knee testing apparatus of claim 13, wherein the positioning system includes a guide with at least one guide rod with a first end attached to a base of the positioning system and a second end slidably coupled to the patient support, the guide permitting the robot to move relative to the patient support in a horizontal direction.

16. A robotic knee testing apparatus of claim 13, wherein the vertical position of the robot is adjustable to permit a knee of the patient to be in flexion in a range of 0 degrees to 90 degrees.

17. A robotic knee testing apparatus of claim 13, wherein the positioning system further includes a column lift with first and second column segments that are telescopically connected to one another to adjust the vertical position of the robot.

18. A robotic knee testing apparatus of claim 13, wherein positioning system further includes a guide permitting the robot to move relative to the patient support in a horizontal direction, and a locking mechanism to lock the positioning system in a selected horizontal position so as to fix the horizontal position of the robot relative to the patient support.

19. A knee examination method comprising the steps of:
positioning a patient adjacent a robotic knee testing apparatus having a patient support and a robot;
moving a positioning system that supports the robot in a horizontal direction away from the patient support to create a gap between a distal edge of the patient support and the robot;
situating the patient within the gap so that the patient can climb onto the patient support; and
adjusting a horizontal position of the robot relative to the patient support using the positioning system to close the gap.

20. A knee examination method of claim 19, further comprising the steps of:
repositioning the patient in a supine position on the patient support; and locating a knee stabilizer of the robot under a knee of the patient to support the knee.

21. A knee examination method of claim 19, further comprising the steps of:
engaging a foot of the patient in a foot plate of the robot; and
further adjusting a vertical position of the robot relative to the patient support to place the knee of the patient in a desired degree of flexion.

22. A knee examination method of claim 21, wherein the step of further adjusting results in flexion of the knee in a range of 0 degrees to 90 degrees.
